(19) United States
(12) Patent Application Publication
(10) Pub. No.: US 2026/0266823 A1
(43) Pub. Date: Sep. 10, 2026

(54) CIRCULATING AUTOANTIBODY BIOMARKERS TO SUPPLEMENT LUNG CANCER SCREENING PROTOCOLS

(71) Applicant: Rush University Medical Center, Chicago, IL (US)

(72) Inventors: Jeffrey Allen Borgia, Flossmoor, IL (US); Claire Auger, Chicago, IL (US)

(21) Appl. No.: 19/166,650

(22) PCT Filed: Mar. 20, 2024
(86) PCT No.: PCT/US2024/020754

§ 371 Date: Sep. 18, 2025

Related U.S. Application Data
(60) Provisional application No. 63/453,250, filed on Mar. 20, 2023.

(51) Int. Cl. G01N 33/5752

(52) U.S. Cl. CPC G01N 33/5752

(57) ABSTRACT

Methods and autoantibody biomarker panels are disclosed that are suitable for detecting and differentiating an actionable lung nodule from a non-actionable lung nodule in a subject using a biological sample obtained from the subject.

### RELATED APPLICATION

This application claims the benefit of priority to U.S. Provisional Application 63/453,250, filed Mar. 20, 2023. The contents of which are incorporated herein by reference in their entirety.

### BACKGROUND

Lung cancer is the leading cause of cancer-related mortality, largely due to late diagnosis. For example, almost half of all non-small cell lung cancer (NSCLC) cases are not detected until after metastasis. While low-dose computer tomography (LDCT) scans have a sensitivity of about 93.7%, they are plagued with a high false positivity rate. Only 3.6% of the initial positive scans are eventually, classified as lung malignancies. Additionally, LDCT is only offered to limited high-risk patients based on relatively strict eligibility criteria prescribed by the U.S. Preventative Task Force (USPTF). Specifically, those with about a 20 cigarettes per day per year smoking history, between the ages of 50-80, and smoking status (i.e., either current smokers or those who have quit within the last 15 years). Based on smoking status alone, it is estimated that at least half of the patients diagnosed with lung cancer would not qualify for screening. Compounding this issue, only about 4-14% of eligible participants attend annual LDCT screenings. Screening rates can vary widely based on where a patient is located and their socioeconomic status. A need exists for a simple test that can confidently identify individuals who would benefit from screening and reduce the false positive rate.

Autoantibodies are antibodies produced by the host's immune system against its own biomolecules. Recently, scientists have been exploring whether autoantibodies could be used as biomarkers for particular diseases. To avoid autotoxicity, a host's B-cells generally have a central and peripheral tolerance, which prevents them from producing autoantibodies targeted at self-antigens. However, it is hypothesized that B-cells can overcome their peripheral tolerance when tumors produce autoantigens that are overly expressed, expressed in areas of the body they typically are not, or are a result of a mutation that leads to an antigenic (or neoantigenic) protein structure. Thus, studies have noted the presence of autoantibodies within cancer and at very early stages of tumor development, making them ideal screening biomarker candidates. With this in mind, a need exists for a simple test to differentiate those high-risk subjects from those who would benefit from LDCT screening and those who would not benefit.

### SUMMARY

The disclosure herein takes advantage of the recent discovery that augmented levels or concentrations of certain autoantibodies in high-risk populations are capable of identifying an actionable lung nodule. By using an autoantibody biomarker panel comprising one or more target proteins specific to the autoantibodies of interest, a subject may provide a sample for detection or diagnosis. Once a subject is diagnosed with an actionable lung nodule, the subject may then be referred to LDCT screening to identify the location and size of the actionable lung nodule. After screening is performed, the subject may undergo a biopsy and/or be administered a treatment for lung cancer.

In some aspects of the disclosure, a method of detecting an actionable lung nodule in a subject is disclosed. In some embodiments, the method comprises contacting a biological sample obtained from the subject comprising an autoantibody biomarker with a target protein; detecting the binding of the target protein with the autoantibody biomarker which is specific to the target protein; and determining a level of the autoantibody biomarker present in the biological sample; wherein the level is indicative of an actionable lung nodule when compared to a reference sample. In another aspect, a method of diagnosing an actionable lung nodule in a subject is disclosed. In some embodiments, the method comprises contacting a biological sample obtained from the subject comprising an autoantibody biomarker with a target protein; detecting the binding of the target protein with the autoantibody biomarker which is specific to the target protein; determining a level of the bound autoantibody biomarker present in the biological sample; and providing a diagnosis based on the level of the bound autoantibody biomarker, wherein the level is indicative of an actionable lung nodule when the level is altered compared to a reference sample. In some aspects, the method of diagnosing an actionable lung nodule in a subject comprises contacting a biological sample obtained from the subject comprising an autoantibody biomarker with a target protein, which is specific to the autoantibody, to form a bound autoantibody; contacting the bound autoantibody with a detection molecule; determining a level of the bound autoantibody present in the biological sample; and providing a diagnosis based on the level of the bound autoantibody biomarker compared to a reference sample, wherein an altered level is indicative of an actionable lung nodule.

In some embodiments, the method further comprises contacting the bound autoantibody with a detection molecule.

In some embodiments, an autoantibody biomarker panel comprising N target proteins, wherein N=1 to 26, and wherein each target protein is specific to an autoantibody for the detection of an actionable lung nodule.

In some embodiments, a kit is disclosed comprising an autoantibody biomarker panel for detecting or diagnosing an actionable lung nodule in a subject.

### BRIEF DESCRIPTION OF THE DRAWINGS

The concepts described herein are illustrative by way of example and not by way of limitation in the accompanying figures.

FIG. 1 shows a whisker box plot showing normalized expression of GPBP1 in lung nodules from actionable verses high risk groups.

FIG. 2 shows a whisker box plot showing normalized expression of HNRNPD in lung nodules from actionable verses high risk groups.

FIG. 3 shows a whisker box plot showing normalized expression of NAT9 in lung nodules from actionable verses high risk groups.

FIG. 4 shows a whisker box plot showing normalized expression of PNMA1 in lung nodules from actionable verses high risk groups.

FIG. 5 shows a whisker box plot showing normalized expression of RAB27A in lung nodules from actionable verses high risk groups.

FIG. 6 shows a whisker box plot showing normalized expression of TAF10 in lung nodules from actionable verses high risk groups.

FIG. 7 shows a whisker box plot showing normalized expression of UBQLN2 in lung nodules from actionable verses high risk groups.

FIG. 8 shows a whisker box plot showing normalized expression of ZNF696 in lung nodules from actionable verses high risk groups.

FIG. 9 shows a whisker box plot showing levels of Annexin 2 in picogram per milliliter (pg/mL) from actionable versus non-actionable node cohorts.

FIG. 10 shows a whisker box plot showing levels of GPBP1 in picogram per milliliter (pg/mL) from actionable versus non-actionable node cohorts.

FIG. 11 shows a whisker box plot showing levels of HNRNPD in picogram per milliliter (pg/mL) from actionable versus non-actionable node cohorts.

FIG. 12 shows a whisker box plot showing levels of IMPDH2 in picogram per milliliter (pg/mL) from actionable versus non-actionable node cohorts.

FIG. 13 shows a whisker box plot showing levels of MED21 in picogram per milliliter (pg/mL) from actionable versus non-actionable node cohorts.

FIG. 14 shows a whisker box plot showing levels of MID1IP1 in picogram per milliliter (pg/mL) from actionable versus non-actionable node cohorts.

FIG. 15 shows a whisker box plot showing levels of PGAM1 in picogram per milliliter (pg/mL) from actionable versus non-actionable node cohorts.

FIG. 16 shows a whisker box plot showing levels of PNMAI in picogram per milliliter (pg/mL) from actionable versus non-actionable node cohorts.

FIG. 17 shows a whisker box plot showing levels of SGPL1 in picogram per milliliter (pg/mL) from actionable versus non-actionable node cohorts.

FIG. 18 shows a whisker box plot showing levels of ZNF696 in picogram per milliliter (pg/mL) from actionable versus non-actionable node cohorts.

FIG. 19 shows a table displaying immunoreagents used in biomarker development.

FIG. 20 shows a table displaying basic clinical information of samples comprising the ‘Discovery’Cohorts used for the HuProt™ protein microarray studies.

FIG. 21 shows a table displaying each target used for which Luminex® assay was developed and used to test the Biomarker Cohort listed.

FIG. 22 shows a table displaying patient demographics of the cohort used to test for the 28 potential biomarkers via Luminex® assays.

FIG. 23 shows a table displaying the breakdown of staging of lung malignancy cases within the training, validation 1, and validation 2 cohorts.

FIG. 24 shows a table displaying individual biomarker performances were compared between “actionable” and “non-actionable” nodules via Mann-Whitney U Test.

FIG. 25 shows a table displaying breakdown of generalized linear model for the ‘top 5’ biomarkers.

FIG. 26 shows a table displaying the breakdown of tumor stages for the malignancy samples which were included in the training, validation 1, and validation 2 cohorts.

FIG. 27 shows a table displaying the performance of sensitivity optimized model broken down by clinically distinct cohort.

### DETAILED DESCRIPTION

The methods and panels disclosed herein, may be used to detect or diagnose an actionable lung nodule or a non-actionable lung nodule. An actionable lung nodule may be indicative of a lung disease, such as lung cancer. The methods and panels disclosed herein take advantage of the discovery that actionable lung nodules are associated with an altered level of expression of autoantibodies in the subject.

### Definitions

The term “actionable” refers to a lung nodule or lesion that is categorized as a 3, 4A, 4B, or 4X on the Lung-RADS® v 1.1 released 2019 and/or Lung-RADS® v 2022 scoring system prepared by the American College of Radiology, the entire contents of which are incorporated herein by reference. Briefly, the lung nodule is at least about 6 mm or greater on its longest axis and has the potential to become active cancer or is suspected of being an active cancer; thus, requiring further diagnostic work-up.

The term “non-actionable” refers to a lung nodule or lesion that is categorized as a 1 or 2 on the Lung-RADS® v1.1 released 2019 and/or Lung-RADS® v2022 scoring system prepared by the American College of Radiology. Briefly, if a lung nodule is present, the lung nodule is less than about 6 mm on its longest axis and is benign and considered to be a low risk of being an active cancer.

The term “autoantibody” refers to an antibody made against biomolecules (e.g., proteins, peptides, etc.) formed by a subject's own body. Autoantibodies have been studied in connection to autoimmune diseases such as arthritis and Grave's disease.

The term “biomarker” refers to any biological compound that can be measured as an indicator of the physiological status of a biological system. Specific to this disclosure, the biomarkers described herein are autoantibody biomarkers that when detected at certain levels are indicative on an actionable lung nodule.

The term “measuring” or “measurement” refers to assessing the presence, absence, quantity or amount (which can be an effective amount) of a given substance within a sample, including the derivation of qualitative or quantitative concentration levels of such substances, or otherwise evaluating the values or categorization of a subject's clinical parameters. Alternatively, the term “detecting” or “detection” may be used and is understood to cover all measuring or measurement as described herein The term “level” or “concentration” refers to the amount measured or detected.

The term “lung nodule” or “lesion” refers to a single mass of abnormal growth in the lungs. The mass may be benign (noncancerous) or malignant (cancerous).

The terms “sample” or “biological sample” as used herein, refers to a sample of biological fluid, tissue, or cells, in a healthy and/or pathological state obtained from a subject. Such samples include, but are not limited to, whole blood, bronchial lavage fluid, sputum, saliva, urine, amniotic fluid, lymph fluid, tissue or fine needle biopsy samples, peritoneal fluid, cerebrospinal fluid, and includes supernatant from cell lysates, lysed cells, cellular extracts, and nuclear extracts. In some embodiments, the whole blood sample is further processed into serum or plasma samples. In some embodiments, the sample includes blood spotting tests.

The term “subject” or “patient” refers to a mammal and preferably a human.

The term “U.S. Preventative Services Task Force” or “USPSTF” refers to an independent group of medical professionals that makes evidence-based recommendations about preventive services such as screenings, behavioral counseling, and preventive medications. Task Force recommendations are created for primary care professionals. The USPSTF issued a Final Recommendation Statement on Lung Cancer Screening on Mar. 9, 2021. The recommendation states that those adults between the ages of 50 to 80 years of age, who have a 20 pack-year smoking history, and who currently smoke or quit within the last fifteen years should seek annual LDCT screenings.

The methods and panels disclosed herein may be used to detect an actionable lung nodule in a subject. An actionable lung nodule may be indicative of a lung cancer as described by the Lung-RADS® v1.1 and v2022. In some embodiments, the lung cancer is a primary lung cancer. In some embodiments, the lung cancer is a secondary cancer having originated from another location. The lung cancer may be non-small cell lung cancer (NSCLC). In some embodiments, the lung cancer is selected from the group consisting of carcinoid, adenocarcinoma (AdCa) and squamous cell carcinoma (SqCC). The presence of an autoantibody biomarker may be indicative of an actionable lung nodule. In some embodiments the presence of more than one autoantibody biomarker may be indicative of an actionable lung nodule. Alternatively, a pattern of expression by two or more autoantibody biomarkers may be indicative of an actionable lung nodule. The expression or pattern of expression of one or more autoantibody biomarkers may differentiate the actionable lung nodule from a non-actionable lung nodule.

### Autoantibody Biomarkers

The methods and panels disclosed herein comprise at least one autoantibody biomarker. The presence and/or measured concentration of the at least one autoantibody biomarker may be used to detect or diagnose an actionable lung nodule in a subject. Without being limited by theory, it is believed that autoantibodies are produced by early and established tumors; and therefore, may be a useful biomarker to diagnose a cancer. As tumors are unregulated environments overexpressing proteins or forming mutated proteins, it may not be possible to identify each autoantibody by a specific amino acid sequence. However, using commercially available assays, as described in more detail herein, it is possible to isolate and identify autoantibodies specifically through the use of target proteins and optionally detection molecules.

For clarity, the autoantibody biomarkers described herein will be identified based on the specific target proteins they each bind to. For example, an autoantibody biomarker may have a binding affinity to a target protein selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, MIDIP1, DCD, MED21, TAF10, ZNF696, DR1, HSP70, GPBP1, MYBPH, PGAM1, IKZF5, NAT9, PNMA1, RAB27A, SGPL1, and TP53. For example, a biomarker may be described as an autoantibody having binding activity to target protein ANNEXIN 2, or the autoantibody biomarker maybe referred to as an ANNEXIN 2 biomarker.

The method may include one or more target proteins. In some embodiments, the one or more target proteins comprise ANNEXIN 2. In some embodiments, the one or more target proteins comprise KEAP1. In some embodiments, the one or more target proteins comprise HNRNPD. In some embodiments, the one or more target proteins comprise IMPDH2. In some embodiments, the one or more target proteins comprise NAPIL5. In some embodiments the one or more target proteins comprise UBIQUILLIN 1. In some embodiments the one or more target proteins comprise UBIQUILLIN 2. In some embodiments, the one or more target proteins comprise ANNEXIN 1. In some embodiments, the one or more target proteins comprise NIP30. In some embodiments, the one or more target proteins comprise CFAP36. In some embodiments, the one or more target proteins comprise MID1P1. In some embodiments, the one or more target proteins comprise DCD. In some embodiments, the one or more target proteins comprise MED21. In some embodiments, the one or more target proteins comprise TAF10. In some embodiments, the one or more target proteins comprise ZNF696. In some embodiments, the one or more target proteins comprise DR1. In some embodiments, the one or more target proteins comprise HSP70. In some embodiments, the one or more target proteins comprise GPBP1. In some embodiments the one or more target proteins comprise MYBPH. In some embodiments, the one or more target proteins comprise PGAM1. In some embodiments, the one or more target proteins comprise IKZF5. In some embodiments, the one or more target proteins comprise NAT9. In some embodiments, the one or more target proteins comprise PNMA1. In some embodiments, the one or more target proteins comprise RAB27A. In some embodiments, the one or more target proteins comprise SGPL1. In some embodiments, the one or more target proteins comprise TP53.

In some embodiments, the one or more target proteins include ANNEXIN 2 and at least one other target protein selected from the group consisting of KEAP1, HNRNPD, IMPDH2, NAP1L5, UBIQUILLIN 1, UBIQUILLIN 2, ANNEXIN 1, NIP30, CFAP36, MIDIP1, DCD, MED21, TAF10, ZNF696, DR1, HSP70, GPBP1, MYBPH, PGAM1, IKZF5, NAT9, PNMA1, RAB27A, SGPL1, and TP53.

In some embodiments, the one or more target proteins include MID1P1 and at least one other target protein selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, DCD, MED21, TAF10, ZNF696, DR1, HSP70, GPBP1, MYBPH, PGAM1, IKZF5, NAT9, PNMA1, RAB27A, SGPL1, and TP53.

In some embodiments, the one or more target proteins include DCD and at least one other target protein selected from the group consisting of ANNEXIN 2, KEAP1, HNRNPD, IMPDH2, NAPIL5, UBIQUILLIN 1, UBIQUILLIN 2, ANNEXIN 1, NIP30, CFAP36, MID1P1, MED21, TAF10, ZNF696, DR1, HSP70, GPBP1, MYBPH, PGAM1, IKZF5, NAT9, PNMA1, RAB27A, SGPL1, and TP53.

In some embodiments, the one or more target proteins include TAF 10 and at least one other target protein selected from the group consisting of ANNEXIN 2, KEAP1, HNRNPD, IMPDH2, NAPIL5, UBIQUILLIN 1, UBIQUILLIN 2, ANNEXIN 1, NIP30, CFAP36, MIDIP1, DCD, MED21, ZNF696, DR1, HSP70, GPBP1, MYBPH, PGAM1, IKZF5, NAT9, PNMA1, RAB27A, SGPL1, and TP53.

In some embodiments, the one or more target proteins include ZNF696 and at least one other target protein selected from the group consisting of ANNEXIN 2, KEAP1, HNRNPD, IMPDH2, NAPIL5, UBIQUILLIN 1, UBIQUILLIN 2, ANNEXIN 1, NIP30, CFAP36, MID1P1, DCD, TAF10, MED21, DR1, HSP70, GPBP1, MYBPH, PGAM1, IKZF5, NAT9, PNMA1, RAB27A, SGPL1, and TP53.

In some embodiments, the one or more target proteins include PNMA1 and at least one other target protein selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, MID1P1, DCD, TAF10, MED21, ZNF696, DR1, HSP70, GPBP1, MYBPH, PGAM1, IKZF5, NAT9, RAB27A, SGPL1, and TP53.

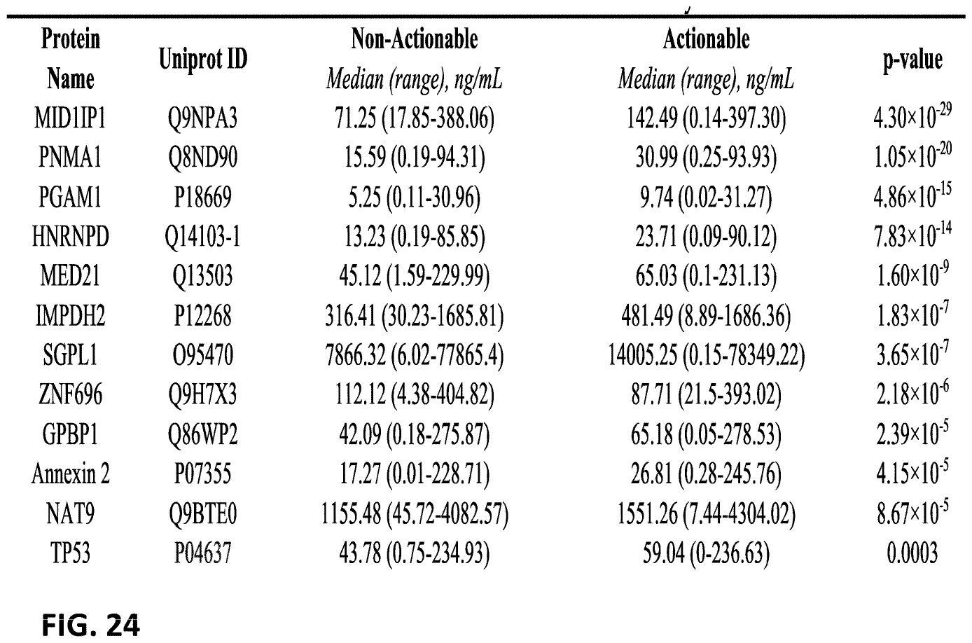

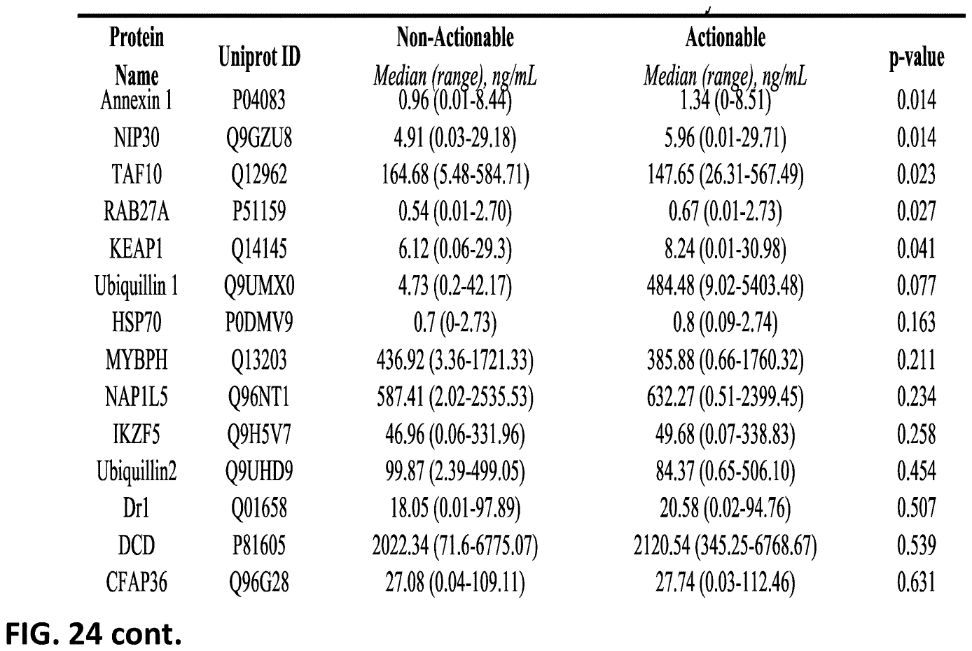

Each target protein identified herein (i.e., ANNEXIN 2, KEAP1, HNRNPD, IMPDH2, NAP1L5, UBIQUILLIN 1, UBIQUILLIN 2, ANNEXIN 1, NIP30, CFAP36, MID1P1, DCD, MED21, TAF10, ZNF696, DR1, HSP70, GPBP1, MYBPH, PGAM1, IKZF5, NAT9, PNMA1, RAB27A, SGPL1, and TP53), may be further characterized by its Uniprot Identification Number. Each Uniprot Identification number is listed in a table provided in FIG. 24. Further, because the biomarkers are autoantibodies, the antigens are human versions of each protein. The target proteins may be provided as full length recombinant proteins, fragments of recombinant proteins, or native (endogenous) proteins.

### Autoantibody Biomarker Panel

In some embodiments, one or more autoantibody biomarkers may be measured using an autoantibody biomarker panel or “panel.” The panel may include a plurality of protein targets to capture and detect the autoantibody biomarkers. In some embodiments, the panel contains N target proteins, wherein N=1 to 26. In some embodiments, the panel may include 26 or fewer target proteins. In yet other embodiments, the panel may include at least one 1, at least 2, at least 3, at least 4, at least 5, or at least 6 target proteins. In some embodiments, the panel may be optimized from a candidate pool of autoantibody biomarkers. By way of a non-limiting example, the panel may be configured for determining whether a high-risk subject has an actionable lung nodule. In some embodiments, the panel may be configured for determining whether a high-risk subject has an actionable lung nodule or a non-actionable lung nodule.

In some embodiments, the panel of target proteins is configured to detect the presence and level of an autoantibody having a binding affinity for ANNEXIN 2. In some embodiments, the panel is configured to detect the presence and level of an autoantibody having a binding affinity for KEAP1. In some embodiments, the panel is configured to detect the presence and level of an autoantibody having a binding affinity for HNRNPD. In some embodiments, the panel is configured to detect the presence and level of an autoantibody having a binding affinity for IMPDH2. In some embodiments, the panel is configured to detect the presence and level of an autoantibody having a binding affinity for NAP1L5. In some embodiments, the panel is configured to detect the presence and level of an autoantibody having a binding affinity for UBIQUILLIN 1. In some embodiments, the panel is configured to detect the presence and level of an autoantibody having a binding affinity for UBIQUILLIN 2. In some embodiments, the panel is configured to detect the presence and level of an autoantibody having a binding affinity for ANNEXIN 1. In some embodiments, the panel is configured to detect the presence and level of an autoantibody having a binding affinity for NIP30. In some embodiments, the panel is configured to detect the presence and level of an autoantibody having a binding affinity for CFAP36. In some embodiments, the panel is configured to detect the presence and level of an autoantibody having a binding affinity for MID1P1. In some embodiments, the panel is configured to detect the presence and level of an autoantibody having a binding affinity for DCD. In some embodiments, the panel is configured to detect the presence and level of an autoantibody having a binding affinity for MED21. In some embodiments, the panel is configured to detect the presence and level of an autoantibody having a binding affinity for TAF10. In some embodiments, the panel is configured to detect the presence and level of an autoantibody having a binding affinity for ZNF696. In some embodiments, the panel is configured to detect the presence and level of an autoantibody having a binding affinity for DR1. In some embodiments, the panel is configured to detect the presence and level of an autoantibody having a binding affinity for HSP70. In some embodiments, the panel is configured to detect the presence and level of an autoantibody having a binding affinity for GPBP1. In some embodiments, the panel is configured to detect the presence and level of an autoantibody having a binding affinity for MYBPH. In some embodiments, the panel is configured to detect the presence and level of an autoantibody having a binding affinity for PGAM1. In some embodiments, the panel is configured to detect the presence and level of an autoantibody having a 11 binding affinity for IKZF5. In some embodiments, the panel is configured to detect the presence and level of an autoantibody having a binding affinity for NAT9. In some embodiments, the panel is configured to detect the presence and level of an autoantibody having a binding affinity for PNMA1. In some embodiments, the panel is configured to detect the presence and level of an autoantibody having a binding affinity for RAB27A. In some embodiments, the panel is configured to detect the presence and level of an autoantibody having a binding affinity for SGPL1. In some embodiments, the panel is configured to detect the presence and level of an autoantibody having a binding affinity for TP53.

The panel may comprise ANNEXIN 2 and at least one other target protein selected from the group consisting of MID1P1, DCD, TAF10, ZNF696, and PNMA1. In some embodiments, the panel comprises ANNEXIN 2, MID1P1, and at least one other target protein selected from the group consisting of DCD, TAF10, ZNF696, and PNMA1. In some embodiments, the panel comprises ANNEXIN 2, MIDIP1, DCD, and at least one other target protein selected from the group consisting of TAF10, ZNF696, and PNMA1. In some embodiments, the panel comprises ANNEXIN 2, MID1P1, DCD, TAF10, and at least one other target protein selected from the group consisting of ZNF696, and PNMA1. The panel may comprise ANNEXIN 2, MIDIP1, DCD, TAF10, ZNF696, and PNMA1.

In some embodiments, the panel may be selected using a reference profile that can be made in conjunction with a statistical algorithm used with a computer to implement the statistical algorithm to sort the subject into a group. In some embodiments, the statistical algorithm is a learning statistical classifier system. The learning statistical classifier system can be selected from the following list of non-limiting examples, including Random Forest (RF), Classification and Regression Tree (CART), boosted tree, neural network (NN), support vector machine (SVM), general chi-squared automatic interaction detector model, interactive tree, multiadaptive regression spline, machine learning classifier, and combinations thereof. By way of a non-limiting example, exemplary tools for selecting a biomarker panel may be found in WO 2012/054732, the contents of which are incorporated herein by reference in their entirety.

### Method of Detection or Method of Diagnosing an Actionable Lung Nodule

Disclosed herein are methods of detecting an actionable lung nodule in a subject. Also disclosed herein are method of diagnosing an actionable lung nodule in a subject. In some embodiments, a method of detecting an actionable lung nodule in a subject comprises contacting a biological sample obtained from the subject comprising an autoantibody biomarker with a target protein; detecting the binding of the target protein with the autoantibody biomarker which is specific to the target protein; determining a level of the autoantibody biomarker present in the biological sample; wherein the level is indicative of an actionable lung nodule when compared to a reference sample.

In some embodiments, a method of diagnosing an actionable lung nodule in a subject comprises contacting a biological sample obtained from the subject comprising an autoantibody biomarker with a target protein, detecting the binding of the target protein with the autoantibody biomarker which is specific to the target protein; determining a level of the bound autoantibody biomarker present in the biological sample; and providing a diagnosis based on the level of the bound autoantibody biomarker, wherein the level is indicative of an actionable lung nodule when the level is altered compared to a reference sample.

The subject may be a human who is classified as high-risk by the USPSTF. In some embodiments, the subject is a smoker. In some embodiments, the subject is a non-smoker but has been exposed to environmental hazards such as asbestos or silica. In some embodiments, the subject is at least about 50 years of age, between the ages of about 50 to about 80 years of age, or up to about 80 years of age. In some embodiments, the subject has been referred to clinic to receive an LDCT scan. The subject may have or be suspected of having lung cancer. The subject may have been graded as a Lung-RADS® v 1.1 or v 2022 score of 1 or 2 in the past. The subject may have been graded as a Lung-RADS® v 1.1 or v 2022 score of 3, 4A, 4B, or 4X in the past. The subject may have a familial history of lung cancer.

The methods may further comprise contacting an autoantibody with a target protein to form a bound autoantibody. In some embodiments, the method further comprises contacting the bound autoantibody with a detection molecule. The detection molecule may be selected from a secondary antibody, a peptide, an aptamer, or the like as those of skill in the art will understand the option for a detection molecule that binds to an antibody. In some embodiments, the detection molecule includes a label. The label may be any molecular tool that produces a signal such as a fluorophore. Detecting the binding of a target protein to an autoantibody biomarker specific to the target protein maybe performed using a detection method selected from using a detection method selected from mass spectrometry, immunoassay, electrochemical luminescent assay, electrochemical voltametry, electrochemical amperometry, atomic force microscopy, radio frequency multipolar resonance spectroscopy, confocal microscopy, non-confocal microscopy, fluorescence optical detection, luminescence optical detection, chemiluminescence optical detection, absorbance optical detection, reflectance optical detection, transmittance optical detection, birefringence optical detection, refractive index detection, surface plasmon resonance, ellipsometry, resonant mirror detection, grating coupler waveguide detection, interferometry, or a combination thereof.

The level of expression generally relates to a quantitative measurement of an expression product which is typically a protein or polypeptide. The disclosure contemplates determining the level or concentration of expression at the RNA (pre-transitional) or protein level (which may include post-translational modification). In particular, the disclosure contemplates determining changes in autoantibody biomarker concentrations reflected in an increase or decrease in the level of transcription, translation, post-transcriptional modification, or the extent or degree of degradation of protein, where these changes are associated with a particular disease state or disease progression.

Samples are collected to ensure that the level of expression in a subject is proportional to the concentration of the autoantibody biomarker in the biological sample. Measurements are made so that the measured value is proportional to the concentration of the autoantibody biomarker in the sample. Thus, the measured value is proportional to the level of expression. Selecting sampling techniques and measurement techniques which meet these requirements is within the skill of the art.

Typically, the level of expression of at least one autoantibody biomarker indicative of an actionable lung nodule differs by a statistically significant degree from the average expression level in normal individuals or the expression levels in subjects with a non-actionable lung nodule; in other words, at least one autoantibody biomarker is statistically deviant from the normal. Statistical significance and deviation may be determined using any known method for comparing means of populations or comparing a measured value to the mean value for a population. Such methods are described in the Examples Section and include the Student's t tests for single and multiple markers considered together, analysis of variance (ANOVA), etc.

As an alternative to, or in combination with determining the level of expression, methods described herein involve determining whether the level of an autoantibody biomarker falls within a normal level (e.g., range) or is outside the normal level (i.e. abnormal). Those who measure levels of biological molecules in biological samples routinely determine the normal level of a particular biomarker in the population they regularly measure, typically described as the normal range of values as determined by the particular laboratory. Thus, the skilled person will be familiar with normal levels of a particular biomarker and can determine whether the level of the autoantibody biomarker is outside of the normal level or range.

More typically, the level of expression of at least one autoantibody biomarker indicative of an actionable lung nodule also differs by a magnitude sufficient such that the differences are analytically significant from the average expression level in normal individuals such that a diagnosis, prognosis, and/or assessment of an actionable lung nodule may be determined. Those of skill in the art understand that greater differences in magnitude are preferred to assist in the diagnosis, prognosis, and/or assessment of a lung disease. See Instrumental Methods of Analysis, Seventh Edition, 1988.

Many proteins expressed by a normal subject or a subject having a non-actionable lung nodule will be expressed to a greater or lesser extent in subjects having an actionable lung nodule or a disease or condition, such as lung cancer. One of skill in the art will appreciate that most diseases manifest changes in multiple, different biomarkers. As such, an actionable lung nodule may be characterized by a pattern of expression of a plurality of autoantibody biomarkers. Indeed, changes in a pattern of expression for a plurality of biomarkers may be used in various diagnostic and prognostic methods, as well as monitoring, therapy selection, and patient assessment methods. The disclosure provides for such methods. These methods comprise determining a pattern of expression of a plurality of autoantibody biomarkers using a panel of target proteins for a particular physiologic state, or determining changes in such a pattern which correlate to changes in physiologic state, as characterized by any technique for suitable pattern recognition.

Numerous methods of determining the level of expression are known in the art. Means for determining expression include but are not limited to radio-immuno assay, enzyme-linked immunosorbent assay (ELISA), high pressure liquid chromatography with radiometric or spectrometric detection via absorbance of visible or ultraviolet light, mass spectrometric qualitiative and quantitative analysis, western blotting, 1 or 2 dimensional gel electrophoresis with quantitative visualization by means of detection radioactive, fluorescent or chemiluminescent probes or nuclei, antibody-based detection with absorptive or fluorescent photometry, quantitation by luminescence of any of a number of chemiluminescent reporter systems, enzymatic assays, immunoprecipitation or immuno-capture assays, solid and liquid phase immunoassays, protein arrays or chips, DNA arrays or chips, plate assays, assays that use molecules having binding affinity that permit discrimination such as aptamers and molecular imprinted polymers, and any other quantitative analytical determination of the concentration of an autoantibody biomarker by any other suitable technique, instrumental actuation of any of the described detection techniques or instrumentation.

The step of determining the level of expression may be performed by any means for determining expression known in the art, especially those means discussed herein. In preferred embodiments, the step of determining the extent of expression comprises performing an immunoassays with target proteins (recombinant full length or truncated portions of molecule or native proteins) and/or antibodies. In some embodiments, the autoantibody and the target protein it is specific to, bind to form a bound autoantibody. The bound autoantibody may be contacted by a detection molecule (e.g., a secondary antibody having a label). The detection methods may be used to detect and measure the level of bound autoantibody in contact with the detection molecule. The level of expression of an autoantibody biomarker may indicate an actionable lung nodule when the expression level is altered compared to a reference sample. The reference sample may be selected from an individual or a pool of individuals having a non-actionable lung nodule or non-diseased (normal) subjects. An altered expression level may refer to an increase or decrease in expression that is statistically significant compared to a non-actionable lung nodule subject sample or normal subject sample. In some embodiments, expression levels of autoantibody biomarkers that indicate an actionable lung nodule are found in FIG. 24.

The confidence of the detection or diagnosis of an actionable lung nodule may increase when more than autoantibody biomarker is measured. In some embodiments, the method analyzes a biological sample from a subject using a panel comprising two target proteins. The panel may comprise at least two target proteins, at least three target proteins, at least four target proteins, at least five target proteins, at least six target proteins, at least seven target proteins, at least eight target proteins, at least nine target proteins, or at least ten target proteins. The panel may comprise up to 6 target proteins, up to 8 target proteins, up to 10 target proteins, up to 12 target proteins, up to 14 target proteins, up to 16 target proteins, up to 18 target proteins, up to 20 target proteins, up to 22 target proteins, up to 24 target proteins, or up to 26 target proteins.

### Treatments of Actionable Lung Nodule

The detection or diagnosis of an actionable lung nodule may result in a referral for a follow-up screening and/or biopsy. In some embodiments, the method further comprising screening the subject after detection or diagnosis of an actionable lung nodule. The screening may use computer tomography (CT). In some embodiments, the screening is performed using a low-dose computer tomography (LDCT). A subject may be referred for screening about every three months, about every six months, about every nine months, or about every twelve months. The method of detecting or diagnosing an actionable lung nodule in a subject may be performed before screening and/or after screening.

The present disclosure also provides methods of treatment based on the detection or diagnosis of an actionable lung nodule. As used herein, the terms “treating,” “treatment,” “therapy,” and like terms refer to administration of a compound or pharmaceutical composition in an amount effective to prevent, alleviate, or ameliorate one or more symptoms of a disease or condition (i.e., indication) and/or to prolong the survival of the subject being treated.

In some embodiments, a treatment is a cancer treatment. The cancer treatment may be selected from surgery, chemotherapy, radiotherapy, immunotherapy, cancer vaccine, or a combination thereof. Examples of approved chemotherapies for treating lung cancer are cisplatin, carboplatin, pemetrexed, paclitaxel, docetaxel, gemcitabine, and vinorelbine. In some embodiments, the cancer treatment is a radiotherapy or radiation. The radiotherapy may be a high-energy x-ray that is administered in a special clinical setting. The cancer treatment may be an immunotherapy treatment. Examples of immunotherapy treatments are engineered CAR-T cells. Examples and protocols for preparing engineered CAR-T cells can be found in US Patent Publication 2024/0075069, published Mar. 7, 2024, the contents of which are incorporated herein by reference in their entirety.

In carrying out the methods of the present disclosure, an effective amount of a treatment is administered to a subject in need thereof. As used herein, the term “effective amount,” in the context of administration, refers to the amount of a treatment (e.g., compound, radiation, or immunotherapy injection) that when administered to a subject is sufficient to prevent, alleviate or ameliorate one or more symptoms of a disease or condition (i.e., indication) and/or to prolong the survival of the subject being treated. Such an amount should result in no or few adverse events in the treated subject. Similarly, such an amount should result in no or few toxic effects in the treated subject. As those familiar with the art will understand, the amount of a treatment (e.g., a compound or pharmaceutical composition) will vary depending upon a number of factors, including without limitation the type of subject being treated, the subject's age, size, weight, and general physical condition, the disorder associated with the subject, and the dosing regimen being employed in the treatment.

In some embodiments of the present disclosure, an effective amount of a compound of the present disclosure to be delivered to a subject in need thereof can be quantified by determining micrograms of a compound of the present disclosure per kilogram of subject body weight. In some embodiments, the amount of a compound administered to a subject is from about 0.1 to about 1000 milligram (mg) of a compound of the present disclosure per kilogram (kg) of subject body weight. As those of ordinary skill in the art understand multiple doses may be used.

In some embodiments, the administration is carried out one or more times per day. In some embodiments, the administration is carried out one time per day. In some embodiments, the administration is carried out two times per day. In some embodiments, the administration is carried out three times per day. In some embodiments, the administration is carried out four times per day.

### Kit

The present disclosure also provides a kit. In some embodiments, the kit a panel as disclosed herein. As used herein, the term “kit” refers to any manufacture, such as, for example, a package, container, and the like, containing a panel and/or reagents according to the present disclosure. In some embodiments, a panel and/or reagents according to the present disclosure is packaged in a vial, bottle, tube, flask or patch, which may be further packaged within a box, envelope, bag, or the like. In some embodiments, a panel and/or reagents according to the present disclosure is approved by the U.S. Food and Drug Administration or similar regulatory agency in the U.S. or a jurisdiction or territory outside the U.S. for administration to a subject. In some embodiments, the kit includes written instructions for use and/or other indication that a panel and/or reagents according to the present disclosure is suitable or approved for administration to a subject. In some embodiments, the kit includes a dispenser.

In addition to the aspects and embodiments described and provided elsewhere in the present disclosure, the following non-limiting list of embodiments are also contemplated.
1. A method of detecting an actionable lung nodule in a subject comprising:
   contacting a biological sample obtained from the subject comprising an autoantibody biomarker with a target protein,
   detecting the binding of the target protein with the autoantibody biomarker which is specific to the target protein;
   determining a level of the autoantibody biomarker present in the biological sample; wherein the level is indicative of an actionable lung nodule when compared to a reference sample.
2. A method of diagnosing an actionable lung nodule in a subject comprising:
   contacting a biological sample obtained from the subject comprising an autoantibody biomarker with a target protein,
   detecting the binding of the target protein with the autoantibody biomarker which is specific to the target protein;
   determining a level of the bound autoantibody biomarker present in the biological sample;
   providing a diagnosis based on the level of the bound autoantibody biomarker, wherein the level is indicative of an actionable lung nodule when the level is altered compared to a reference sample.
3. The method of clauses 1 or 2, wherein the subject is a human.
4. The method of clauses 1 or 3, wherein the subject is an adult human.
5. The method of clauses 1 to 4, wherein the subject is at least about 50 years of age.
6. The method of clauses 1 to 5, wherein the subject is at most about 80 years of age.
7. The method of clauses 1 to 6, wherein the subject is between the ages of about 50 to about 80 years of age.
8. The method of clauses 1 to 7, wherein the subject is a smoker.
9. The method of clauses 1 to 8, wherein the subject is a former smoker.
10. The method of clauses 1 to 9, wherein the subject has a history of smoking at least one pack of cigarettes a day.
11. The method of clauses 1 to 10, wherein the subject has a history of smoking for at least one year, at least five years, at least ten years, at least 15 years, at least 20 years, or more than 20 years.
12. The method of clauses 1 to 11, wherein the subject is at a high-risk of developing lung cancer.
13. The method of clause 12, wherein the subject is at a high-risk due to environmental exposure or a familial genetic predisposition.
14. The method of clauses 1 to 13, wherein the biological sample is selected from the group consisting of whole blood, plasma, serum, bronchial lavage fluid, sputum, saliva, urine, amniotic fluid, lymph fluid, tissue or fine needle biopsy samples, peritoneal fluid, cerebrospinal fluid, and includes supernatant from cell lysates, lysed cells, cellular extracts, and nuclear extracts.
15. The method of clause 14, wherein the whole blood is collected by a finger prick.
16. The method of clause 15, wherein the whole blood is collected on a blood card.
17. The method of clause 14, wherein the whole blood is collected via phlebotomy.
18. The method of clauses 1-17, wherein the target protein is a full length recombinant protein, a fragment of a recombinant protein, or a native protein.
19. The method of clauses 1-18, wherein the target protein is selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, MID1P1, DCD, MED21, TAF10, ZNF696, DR1, HSP70, GPBP1, MYBPH, PGAM1, IKZF5, NAT9, PNMA1, RAB27A, SGPL1, and TP53.
20. The method of clauses 1 to 19, wherein the target protein is ANNEXIN 2, and the method further comprises at least one other target protein selected from the group consisting of KEAP1, HNRNPD, IMPDH2, NAP1L5, UBIQUILLIN 1, UBIQUILLIN 2, ANNEXIN 1, NIP30, CFAP36, MID1P1, DCD, MED21, TAF10, ZNF696, DR1, HSP70, GPBP1, MYBPH, PGAM1, IKZF5, NAT9, PNMA1, RAB27A, SGPL1, and TP53.
21. The method of clauses 1 to 19, wherein the target protein is MID1P1 and the method further comprises at least one other target protein selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, DCD, MED21, TAF10, ZNF696, DR1, HSP70, GPBP1, MYBPH, PGAM1, IKZF5, NAT9, PNMA1, RAB27A, SGPL1, and TP53.
22. The method of clauses 1 to 18, wherein the target protein is DCD and the method further comprises at least one other target protein selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, MID1P1, MED21, TAF10, ZNF696, DR1, HSP70, GPBP1, MYBPH, PGAM1, IKZF5, NAT9, PNMA1, RAB27A, SGPL1, and TP53.
23. The method of clauses 1 to 19, wherein the target protein is TAF 10 and the method further comprises at least one other target protein selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, MID1P1, DCD, MED21, ZNF696, DR1, HSP70, GPBP1, MYBPH, PGAM1, IKZF5, NAT9, PNMA1, RAB27A, SGPL1, and TP53.
24. The method of clauses 1 to 19, wherein the target protein is ZNF696 and the method further comprises at least one other target protein selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, MID1P1, DCD, TAF10, MED21, DR1, HSP70, GPBP1, MYBPH, PGAM1, IKZF5, NAT9, PNMA1, RAB27A, SGPL1, and TP53.
25. The method of clauses 1 to 19, wherein the target protein is PNMA1, and the method further comprises at least one other target protein selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, MID1P1, DCD, TAF10, MED21, ZNF696, DR1, HSP70, GPBP1, MYBPH, PGAM1, IKZF5, NAT9, RAB27A, SGPL1, and TP53.
26. The method of clauses 1 to 19, wherein the target protein is ANNEXIN and the method further comprises at least one other target protein selected from the group consisting of MID1P1, DCD, TAF10, ZNF696, and PNMA1.
27. The method of clauses 1 to 19, wherein the target protein is MID1P1 and the method further comprises at least one other target protein selected from the group consisting of ANNEXIN 2, DCD, TAF10, ZNF696, and PNMA1.
28. The method of clauses 1 to 19, wherein the target protein is DCD and the method further comprises at least one other target protein selected from the group consisting of ANNEXIN 2, MID1P1, TAF10, ZNF696, and PNMA1.
29. The method of clauses 1 to 19, wherein the at least one biomarker includes an autoantibody having binding activity against the antigen TAF10 and the method further comprises at least one other target protein selected from the group consisting of ANNEXIN 2, DCD, MID1P1, ZNF696, and PNMA1.
30. The method of clauses 1 to 19, wherein the target protein is ZNF696 and the method further comprises at least one other target protein selected from the group consisting of ANNEXIN 2, DCD, TAF10, MID1P1, and PNMA1.
31. The method of clauses 1 to 19, wherein the target protein is PNMA 1 and the method further comprises at least one other target protein selected from the group consisting of ANNEXIN 2, DCD, TAF10, MID1P1, and ZNF696.
32. The method of clauses 1 to 19, wherein the target protein is ANNEXIN 2 and the method further comprises a second target protein of MID1P1.
33. The method of clauses 1 to 19, wherein the target protein is ANNEXIN and the method further comprises a second target protein of DCD.
34. The method of clauses 1 to 19, wherein the target protein is ANNEXIN 2 and the method further comprises a second target protein of TAF10.
35. The method of clauses 1 to 19, wherein the target protein is ANNEXIN 2 and the method further comprises a second target protein of ZNF696.
36. The method of clauses 1 to 19, wherein the target protein is ANNEXIN 2 and the method further comprises a second target protein of PNMA1.
37. The method of clauses 1 to 19, wherein the target protein is MID1P1 and the method further comprises a second target protein of DCD.
38. The method of clauses 1 to 19, wherein the target protein is MID1P1 and the method further comprises a second target protein of TAF10.
39. The method of clauses 1 to 19, wherein the target protein is MID1P1 and the method further comprises a second target protein of ZNF696.
40. The method of clauses 1 to 19, wherein the target protein is MID1P1 and the method further comprises a second target protein of PNMA1.
41. The method of clauses 1 to 19, wherein the target protein is DCD and the method further comprises a second target protein of TAF10.
42. The method of clauses 1 to 19, wherein the target protein is DCD and the method further comprises a second target protein of ZNF696.
43. The method of clauses 1 to 19, wherein the target protein is DCD and the method further comprises a second target protein of PNMA1.
44. The method of clauses 1 to 19, wherein the target protein is TAF 10 and the method further comprises a second target protein of ZNF696.
45. The method of clauses 1 to 19, wherein the target protein is TAF 10 and the method further comprises a second target protein of PNMA1.
46. The method of clauses 1 to 19, wherein the target protein is ZNF696 and the method further comprises a second target protein of PNMA1.
47. The method of clauses 1 to 46, wherein the level is determined using an immunoassay.
48. The method of clauses 1 to 47 further comprising a panel of N target proteins, wherein N=1 to 26, and wherein each target protein is specific to an autoantibody.
49. The method of clause 48, wherein the target proteins are selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, MID1P1, DCD, TAF10, MED21, ZNF696, DR1, HSP70, GPBP1, MYBPH, PGAM1, IKZF5, NAT9, RAB27A, SGPL1, and TP53.
50. The method of clauses 1 to 49, wherein detection of the autoantibody biomarker is performed using a detection method selected from mass spectrometry, immunoassay, electrochemical luminescent assay, electrochemical voltametry, electrochemical amperometry, atomic force microscopy, radio frequency multipolar resonance spectroscopy, confocal microscopy, non-confocal microscopy, fluorescence optical detection, luminescence optical detection, chemiluminescence optical detection, absorbance optical detection, reflectance optical detection, transmittance optical detection, birefringence optical detection, refractive index detection, surface plasmon resonance, ellipsometry, resonant mirror detection, grating coupler waveguide detection, interferometry, or a combination thereof.
51. The method of clauses 1 to 50, wherein reference sample is provided from a pool of healthy individuals or from a pool of non-actionable lung nodule samples.
52. The method of clauses 1 to 51, wherein the actionable lung nodule is indicative of lung cancer.
53. The method of clause 52, wherein the lung cancer is selected from the group consisting of carcinoid, adenocarcinoma (AdCa) and squamous cell carcinoma (SqCC).
54. The method of clauses 1 to 53 further comprising screening the subject.
55. The method of clause 54, wherein the screening is performed using a low-dose computer tomography (LDCT) machine.
56. The method of clauses 1 to 55 further comprising administering a therapeutic effective amount of a cancer treatment.
57. The method of clause 56, wherein the cancer treatment is configured to treat a lung cancer.
58. The method of clauses 56 to 57, wherein the cancer treatment is selected from surgery, chemotherapy, radiotherapy, immunotherapy, cancer vaccine, or a combination thereof.
59. The method of clauses 56 to 58, wherein the cancer treatment is chemotherapy.
60. The method of clauses 56 to 58, wherein the cancer treatment is a radiotherapy.
61. The method of clauses 56 to 58, wherein the cancer treatment is an immunotherapy.
62. An autoantibody biomarker panel comprising N target proteins, wherein N=1 to 26, and wherein each target protein is specific to an autoantibody for the detection of an actionable lung nodule.
63. The panel of clause 62, wherein the target proteins are selected from ANNEXIN 2, KEAP1, HNRNPD, IMPDH2, NAP1L5, UBIQUILLIN 1, UBIQUILLIN 2, ANNEXIN 1, NIP30, CFAP36, MIDIP1, DCD, TAF10, MED21, ZNF696, DR1, HSP70, GPBP1, MYBPH, PGAM1, IKZF5, NAT9, RAB27A, SGPL1, and TP53.
64. A kit comprising a panel described in previous clauses 1-63.
65. The kit of clause 65, further comprising instructions, packaging, and reagents.
66. The methods of any of the preceding clauses further comprising detecting the autoantibody with a detection antibody.
67. The kit of any of the preceding clauses further comprising a detection antibody.
68. A method of detecting an actionable lung nodule in a subject comprising:
   contacting a biological sample obtained from the subject comprising an autoantibody biomarker with a target protein, which is specific to the autoantibody, to form a bound autoantibody;
   contacting the bound autoantibody with a detection molecule;
   detecting the binding of the bound autoantibody with the detection molecule;
   determining a level of the bound autoantibody present in the biological sample and comparing it to a reference sample; wherein an altered level of bound autoantibody is indicative of an actionable lung nodule when compared to a reference sample.
69. A method of diagnosing an actionable lung nodule in a subject comprising:
   contacting a biological sample obtained from the subject comprising an autoantibody biomarker with a target protein, which is specific to the autoantibody, to form a bound autoantibody;
   contacting the bound autoantibody with a detection molecule; determining a level of the bound autoantibody present in the biological sample;
   providing a diagnosis based on the level of the bound autoantibody biomarker compared to a reference sample, wherein an altered level is indicative of an actionable lung nodule.
70. The method of clauses 68 to 69, wherein the detection molecule is configured to bind to an antibody.
71. The method of clause 70, wherein the detection molecule includes a label.
72. The method of clauses 70 to 71, wherein the detection molecule is a secondary antibody.

### EXAMPLES

Examples related to the present disclosure are described below. In most cases, alternative techniques can be used. The examples are intended to be illustrative and are not limiting or restrictive of the scope of the invention as set forth in the claims.

### Example 1

Identifying Autoantibody Biomarker using Protein Microarray and Luminex® Immunobead Platforms: Application-Companion Diagnostics for Lung Cancer Screening.

Lung cancer is the leading cause of cancer-related mortality and despite advancements in CT (computed tomography)-based screening, uptake remains low in the population. Circulating biomarkers fashioned into a simple and cost-effective companion diagnostic test could revolutionize lung cancer screening. We have developed a pipeline for the discovery and development of novel candidate biomarkers to build a companion diagnostic method. This approach relies upon screening for candidate biomarkers using high-density protein microarrays, followed by translation of candidates into Luminex® immunobead platforms, captures and allows for identification of analytes in a sample for further testing. Specifically, we identified novel autoantibody biomarkers capable of discerning between population at “high risk” for lung cancer or with Benign lung lesions (BN) from those with pathologically-diagnosed lung cancer, including carcinoid, adenocarcinoma (AdCa) and squamous cell carcinoma (SqCC).

### Methods

148 serum samples were obtained, accrued from patients with pathologically-diagnosed lung cancer (n=35 AdCa, n=27 SqCC, and n=24 Carcinoid), pathologically-diagnosed non-malignant lesions (n=32 BN), and those at “high-risk” for lung cancer (n=30). Sample pools were created for these specimen sets, sampling each group 2-4 times, averaging 5 samples per pool. Each sample pool was tested using HuProt™ human protein microarrays, a platform which evaluates autoantibody reactivity to over 21,000 targets, following published protocols. Data was normalized across the sample pools and differential comparisons performed using eBayes, empirical Bayes statistics for differentially expression, in R. Candidate biomarkers were selected for Luminex® assay development based on significance levels and/or magnitude of application to possible relevant comparisons [i.e., AdCa vs. High-Risk, AdCa vs. BN, SqCC vs. High-Risk, SqCC vs. BN, carcinoid vs. High-Risk, carcinoid vs. BN, NSCLC (AdCa and SqCC) vs. High-Risk, and NSCLC vs. BN]. Luminex® assays were developed using standard carbodiimide/NHS (N-hydroxysuccinimide) ester chemistry. Analytical assay performance characteristics were established for each assay using an anti-target/PE (phycoerythrin)-conjugated secondary reporter system. Validated assays were used to assess individual patient samples, defined above, with Luminex® results contrasted to those from the HuProt™M microarray.

### Results

501 candidate biomarkers (p≤0.01) were identified by HuProt™ microarray relevant to early diagnosis of lung cancer. From these findings, 8 biomarkers developed into Luminex® assays are featured: TAF10, PNMA1, GPBP1, NAPIL5, NAT9, ZNF696, HNRNPD, and RAB27A. As some of these markers showed significance for more than one comparison (described in Methods), a total of 17 comparisons were significant, which were subsequently re-assessed on the Luminex® platform with individual samples. 12 of 17 comparisons that showed significant discernment via microarray, remained significant (p<. 05) when tested on the Luminex® platform. In those not reaching significance via Luminex®, three showed the same directionality. Interestingly, the three least significant comparisons were markers selected based on higher signal in the control. This suggests that potential markers should be filtered based on elevated levels in the disease groups.

### Differentially Expressed Autoantibodies Found in the Microarray

Through the use of HuProt™ microarrays, autoantibody profiles were determined for three types of Lung Malignancies: Adenocarcinoma (AdCa), Squamous Cell Carcinoma (SqCC), and Carcinoid Tumors, and two cohorts of patients without malignancy: High-Risk Controls and Benign Nodule. The autoantibody profiles of the malignancy cohorts (including NSCLC which is the AdCa and SqCC outputs combined) were compared to the autoantibody profiles of the High-Risk Controls and the Benign Nodule Cohorts, to determine differentially expressed autoantibodies which may serve as good biomarkers. The results of these comparisons identified a total of 501 potential autoantibody targets with significant p-values (<01). Further selection of these markers occurred, with a preference for markers which held significance for more than one comparison, were increased in the malignancy group over the control group and held a significant adjusted p-value. From these findings, 8 of the biomarkers developed into Luminex® Assays are featured: TAF10, PNMA1, GPBP1, NAPIL5, NAT9, ZNF696, HNRNPD, and RAB27A

### Luminex® for the Measurement of Microarray Targets

To determine whether the microarray results would be reflected by Luminex® assays run on the individual serum samples, 8 featured markers were re-measured via Luminex®. For the 8 markers selected a total of 17 different comparisons of interest had differential expression. What we found was that 12/17 comparisons significant via microarrays tested on patient sample pools were still significant (p-value<0.05) when re-assessed via Luminex® assays on the individual serum samples 3/5 not significant showed the same directionality seen in the microarrays, however, did not reach significance. Interestingly, all 3 comparisons assessed where the marker selected was upregulated in the non-malignancy group, were not significant when tested via the Luminex® on the individual samples. This suggests it would be of interest to focus the final selections from the microarray on markers upregulated in the disease group rather than downregulated.

### Conclusions

Herein, we define a convenient workflow for the discovery of candidate autoantibody biomarkers using high-density protein microarrays followed by target validation on the Luminex® platform. Overall, this method shows excellent concordance between differential findings between cohorts on microarray and Luminex® platforms, suggesting this discovery pipeline holds promise for a broad spectrum of potential diagnostic applications.

### Example 2

Development of a Novel Circulating Autoantibody Biomarker Panel for the Identification of Patients with ‘Actionable’ Pulmonary Nodules

Due to poor compliance and uptake of LDCT (low dose computed tomography) screening among high-risk populations lung cancer is often diagnosed in advanced stages where treatment is rarely curative. Although 80-90% of patients screened will have clinically “non-actionable” nodules (LungRADS® (Lung Imaging Reporting and Data System, classification system for lung cancer with 1 being category 1 being no lung nodes and category 4 being high chance of malignant lung nodes) 1 or 2), those harboring larger, clinically “actionable” nodules (LungRADS® 3 or 4) have a significantly greater risk of lung cancer. The development of a companion diagnostic method capable of identifying patients likely to have a clinically actionable nodule identified during LDCT (low dose computed tomography) is anticipated to improve accessibility and uptake of the paradigm and improve early detection rates. Using protein microarrays, we identified 501 circulating targets with differential immunoreactivities against a cohorts characterized as possessing either actionable (n=43) or non-actionable (n=20) solid pulmonary nodules, per LungRADS® guidelines. Quantitative assays were assembled on the Luminex® platform for the 26 most promising targets. These assays were used to measure serum autoantibody levels in 861 patients, consisting of benign (BN; n=101), early-stage non-small cell lung cancer (NSCLC; n=245), and individuals meeting USPTF (United States Preventative Task Force) screening inclusion criteria with negative radiologic findings (n=466). These 861 patients were randomly split into three cohorts: training, validation 1, and validation 2. Of the 26 candidate biomarkers tested, 17 differentiated patients with actionable nodules from those with non-actionable nodules. A random forest model consisting of 6 autoantibody (Annexin 2, DCD, MID1IP1, PNMA1, TAF10, ZNF696) biomarkers was developed to optimize our classification performance, possessing a PPV (positive predictive value) of 61.4%/61.0% and NPV (negative predictive value) of 95.7%/83.9% against validation cohorts 1 and 2, respectively. This panel may improve patient selection methods for lung cancer screening, serving to greatly reduce the futile screening rate while also improving accessibility to the paradigm for underserved populations.

Lung cancer is the leading cause of cancer-related mortality, largely due to late diagnosis. One key contributor to the particularly poor outcomes seen in patients with NSCLC, is that almost half of all NSCLC cases are not detected until after metastasis. While LDCT scans have a sensitivity of 93.7%, they are plagued with a high false positivity rate. Only 3.6% of the initial positive scans are eventually, classified as lung malignancies. LDCT is only offered to limited patients based on relatively strict eligibility criteria, specifically, those with 20 pack-year smoking history, between the ages of 50-80, and smoking status (current smokers or those who have quit within the last 15 years). Based on the smoking status alone, it is estimated that at least half of the patients diagnosed with lung cancer would not qualify for screening. Compounding this issue, among those who are eligible for screening, compliance has been exceptionally low, estimated at 4-14%. Screening rates can vary widely based on where a patient is located and their socioeconomic status.

In the latest screening recommendation, the USPSTF noted a need for biomarkers that can identify patients at high-risk of developing lung cancer and lower the rate of false-positives. To this end, our laboratory identified circulating biomarkers that can be used as molecular indicators to identify individuals likely to have clinically “actionable” solid pulmonary nodules. Actionable nodules, based upon the Lung-RADS® v1.1 and v2022 definition (LungRADS® 3 or 4), consists of individuals with solid pulmonary nodules that are equal to or greater than 6 mm. These patients are considered high risk for lung cancer, and it is recommended that they get screened more frequently or undergo additional testing which is often invasive. One study found that approximately 80.6% of patients had clinically “non-actionable” nodules and had low risk of lung malignancy of <0.1%. This can be compared to a lung malignancy risk of 0.9% in patients with “indeterminate” nodules (26 mm, <8 mm, LungRADS® 3), and 9.6% for those with positive LDCT (28 mm, LungRADS® 4). With a biomarker test which can identify patient populations with “actionable” nodules, we can identify patients who would benefit most from LDCT scans. This method could potentially increase uptake of screening in underserved populations as blood-based test are more readily accessible, serve to ameliorate those with screening hesitancy since the test could be administered in primary care, and help lower false-positive rates.

This study is focused on developing a panel of circulating autoantibody biomarkers to answer this clinical need. We begin with a discovery effort that uses high-throughput protein microarrays to identify candidate biomarkers with differential signal between patients with clinically “actionable” and “non-actionable” nodules. These candidate biomarkers are then assessed in tandem with other potential biomarkers for this application with custom Luminex® assays built ‘in house’ with a larger cohort of patients. Machine learning was then used to identify the optimal combination of autoantibody biomarkers for discerning “actionable” and “non-actionable” nodules, which can serve as a companion risk-stratification method in conjunction with current lung cancer screening protocols.

Materials and Methods The overall methodology can be divided into three steps. These steps will be referred to throughout the material and methods section.

Step 1 involves the discovery of novel candidate biomarkers for discerning “actionable” versus “non-actionable” nodules utilizing HuProt™ microarrays with our “Discovery” cohort (n=63 total samples; divided into 10 sample pools). A total of 26 candidate biomarkers are identified. In Step 2, custom Luminex® immunobead assays are developed for the candidate biomarkers identified in Step 1 and used to assess their performance against our Biomarker Discovery Cohort (n=841). Each marker was statistically evaluated for its individual value for discerning “actionable” versus “non-actionable” nodules. In Step 3 the data from the Biomarker Discovery Cohort (n=841) is split into three cohorts, “training”, “validation 1”, and “validation 2”. The Random Forest algorithm was used to develop a biomarker panel based on the optimal combination of 6 features. Performance characteristics of this model for discerning “actionable” versus “non-actionable” cases was then evaluated and optimized using Validation cohorts 1 and 2.

### Patient Cohorts

All serum samples were obtained from the Rush University Cancer Center Biorepository. All patients enrolled gave written, informed consent prior to bio-specimen collection. Cases denoted as having a lung malignancy or as benign were classified based on a pathological diagnosis of tissue obtained from anatomic resections. The high-risk screening cohort was comprised of patients who qualified for lung cancer screening based on USPSTF guidelines but did not have a lung malignancy at the time of the blood draw.

Two separate cohorts were collected for the purposes of this study. The first is a ‘Discovery’ cohort (n=63) used in our experiments to identify novel candidate biomarkers via HuProt™ microarray. The second cohort we termed the ‘Biomarker Development’ cohort (n=841). This cohort was randomly divided into ‘Training’ (n=565), ‘Validation 1’ (n=93), and ‘Validation 2’ (n=183; aka a ‘test’ cohort) cohorts to permit the development and validation of a multi-analyte panel for classifying patients based on actionable nodule status.

Cases for this study were classified as “actionable” versus “non-actionable” based on nodule sizes annotated in the radiology report by a board-certified radiologist. All cases with solid nodules ≥6 mm were considered actionable, consistent with Lung-RADS® v 1.1 and v2022. Instances of nodules that fell between 4 mm to 6 mm, Lung-RADS® scores were pulled from the radiologist reports and are reflective of the time of screening/sample collection. These Lung-RADS® scores were based on either Lung-RADS® v1.0 or v1.1, depending on the time of screening/specimen collection. Cases which were not classified as “actionable” were classified as “non-actionable”.

### High-density Protein Microarrays (Step 1)

Patient sera from the ‘Discovery’ cohort were evaluated on high-density HuProt™ v4.0 Protein Proteome microarrays by CDI Laboratories (Mayagüez, Puerto Rico). HuProt™ v4.0 Protein Proteome microarrays have been found to be a reproducible mechanism for discovery of autoantibody targets within malignancy samples and has been the basis of other studies looking into the development of machine learning models for malignancy prognosis and diagnosis. A total of 10 microarrays were run on pooled samples for this study that divide into ‘non-actionable’ and ‘actionable’ nodule categories. Non-actionable nodules were assessed as 2 groups (n=10 per group), whereas actionable nodules were assessed as 8 groups total: 4 from Squamous Cell Carcinoma (SqCC; n=3/3/6/5) patients and 4 from Adenocarcinoma (AdCa; n=6/6/6/7) patients.

After protein microarray results were obtained, raw GPR files were converted to a raw excel file which contained the signal intensity for 23,059 proteins (>21,000 of which were unique, with 2,000 technical replicates) for each of the sample pools. To help mitigate any batch effect variation, data was normalized. A total of six different normalization methodologies were utilized to ensure robust results. These included Cyclic-Loess, Log2, trimmed mean of m-values (TMM), and Quantile, and Robust Linear Model (RLM) processed through a widget developed for analyzing protein microarray data, called PAWER. For each of the normalization methods, differential analyses using empirical Bayesian statistics (implemented via the limma package of Bioconductor), were performed comparing the “actionable” cohorts (AdCa, SqCC, or the combined) to the “non-actionable” high-risk screening cohort. Adjusted p-values were calculated using the Benjamini-Hochberg method. Of the 23,059 autoantibodies compared, a total of 501 markers had p-values <0.05, based on the moderated t-tests run comparing the “actionable” cohorts to the “non-actionable” cohorts. From these 501 markers, we identified 8 promising candidate biomarkers for discerning non-actionable against actionable nodules (either as entire category or an individual histological sub-type) and another 10 that were relevant to lung cancer screening. Another 8 biomarkers we previously identified for early lung cancer detection were also selected for evaluation.

### Custom Luminex® Immunobead Assay Development (Step 2)

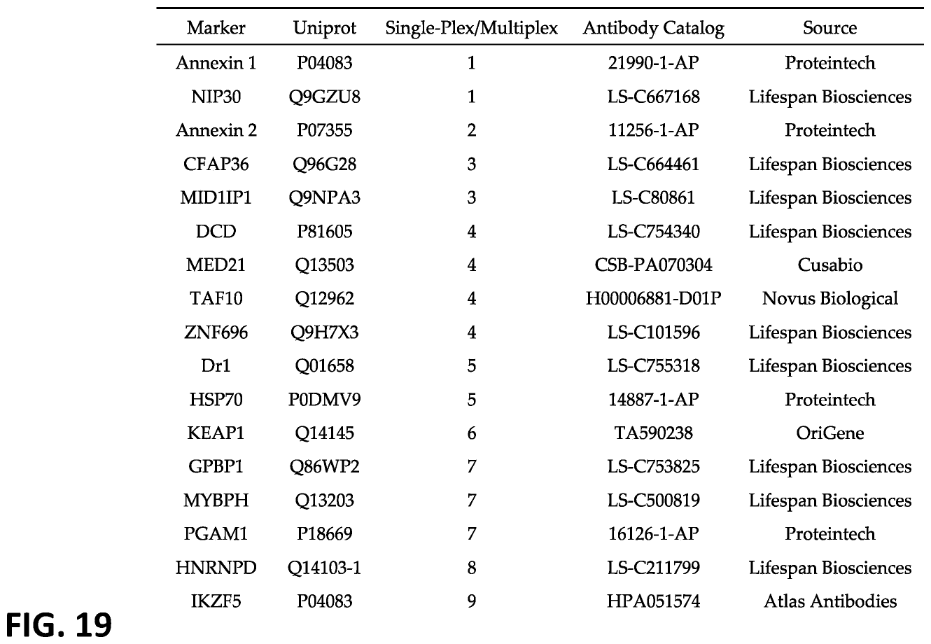

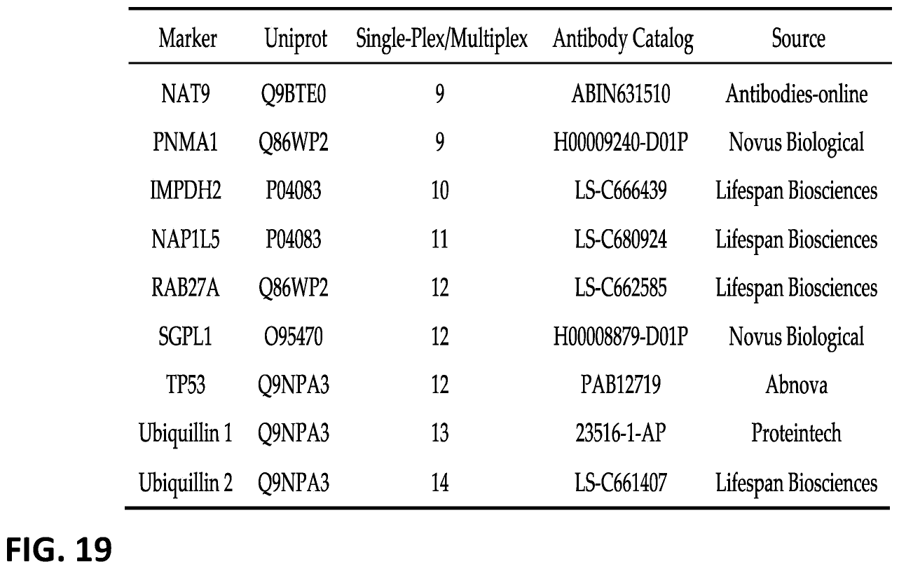

A custom Luminex® immunobead assay was built for each of the 26 selected targets described in the previous section using methods we previously reported. Briefly, assay construction was accomplished upon conjugation of each recombinant protein (a.k.a. target protein or antigen) on a unique MagPlex® bead region, polystyrene beads that allow for measurement of analytes, via standard sulfo-NHS/EDC chemistries. Conjugation efficiencies and (analytical) characteristics for each assay were evaluated against a 7-point standard curve of the corresponding anti-target antibody (rabbit polyclonal); see FIG. 19 Supplemental Table 1 for details. Assay characteristics determinations included ‘working range’ assessments (limits of detection and quantitation), optimal sample dilution determinations, and assessments of performance characteristics (sensitivity, specificity, etc.). Assays were then tested for their ability to be combined into multiplex panels, using ‘leave-one-out’ testing to identify cross-reactivity issues, as we previously described. From these efforts, 14 single-plex or multiplex panels were qualified for specimen testing that were optimized for sample dilution, primary incubation times, and secondary incubation times.

### Cohort Testing (Step 2)

Custom Luminex® assays developed in the previous section for the 26 candidate biomarkers were then used to assess serum from our Biomarker Discovery cohort. The Biomarker Discovery cohort consisted of 841 cases that were either screened by our Diagnostic Radiology Department for lung cancer or received an anatomic resection by our Department of Cardiothoracic Surgery. All samples were processed on 384-well plates with duplicate sampling and had a 7-point standard curve on each plate, as we previously described. Detection of patient autoantibodies was accomplished using PE-conj., rabbit anti human IgG antibodies (as shown in FIG. 24). Each plate was read on a FlexMap 3D (Luminex Corp.), multiplexing platform that allows for analysis of various immunoassays, to obtain median fluorescence intensity (MFI) values in xPonent v4.3 (Luminex Corp.), assay analysis software. Concentrations of each marker for each sample in the large cohort was calculated using Belysa v1.1 Software, curve-fitting software. The software mapped the MFI signal from the sample well to the 4PL logistic curve produced based on the 7-point standard curve of the anti-target antibody. Replicates which had a coefficient of variation equal to or greater than 50%, were removed as were reads which had <30 beads/well, based upon thresholds recommended by Luminex® and in the literature.

### Luminex ® Data Pre-processing and Analysis (Step 2)

Boxplots comparing “actionable” nodule patients to “non-actionable” nodule patients were produced for all biomarkers tested, with p-values determined via Mann-Whitney (two-sided) U tests. Outliers were removed for the creation of the boxplots and for the determination of the individual marker p-values. ROC (receiver operating characteristic curve) and AUC (area under the curve) curves were produced for each of the individual biomarkers utilizing the pROC package in R. The top 10 most significant biomarkers were selected based on the Mann-Whitney U test results. For these biomarkers, a generalized linear model was trained on 70% of the data, with an optimal cut-off selected which produced the highest true positive rate. The developed models were then applied to the remaining 30% of the cohort and performance characteristics were assessed.

### Development of a Multianalyte Panel for Patient Risk Stratification (Step 3)

Prior to any machine learning, data from the Biomarker Discovery cohort was split randomly into 3 separate sets: Training (n=565), Validation 1 (n=93), and Validation 2 (n=183; aka a ‘test’ cohort). Biomarkers with at least 65% non-missing values were considered for panel development.

For the purposes of variable selection, each possible six or seven marker combination of 23 biomarkers was considered. A random forest prediction model is developed for each of the combinations using the “Training” cohort. Prediction performance of each of the random forest models developed were determined based on OOB (out-of-bag) error of the model in the training set and the prediction accuracy of the model in the Validation 1 cohort. The marker combination which resulted in a random forest model with the best performance metrics was selected as the final panel.

The final random forest model was trained using the Training set, based on the 6-marker panel selected. The performance characteristics of this model at classifying “actionable” from “non-actionable” nodules was calculated for both the Validation 1 and Validation 2 cohort.

An optimal cut-off was created to minimize ‘false-negative’ results given that sensitivity was considered paramount to our application. The prediction performances at each actionable “vote” score cut-off, which is the proportion of trees in the model which assessed the case as actionable, in the Validation 1 cohort were used to create an ROC curve. An optimal threshold was determined by detecting a “vote” score cut-off which offered >95% sensitivity in the Validation 1 cohort, while minimizing the amount of ‘false-positives’. This cutoff value was then used to recalculate panel performance characteristics against the Validation 2 cohort. Finally, the performance metrics were further evaluated in the clinically distinct groups for each of the different cohorts tested.

### Results

### Patient Population for the HuProt™ Microarrays for the Discovery of Novel Lung Cancer Early Detection Targets

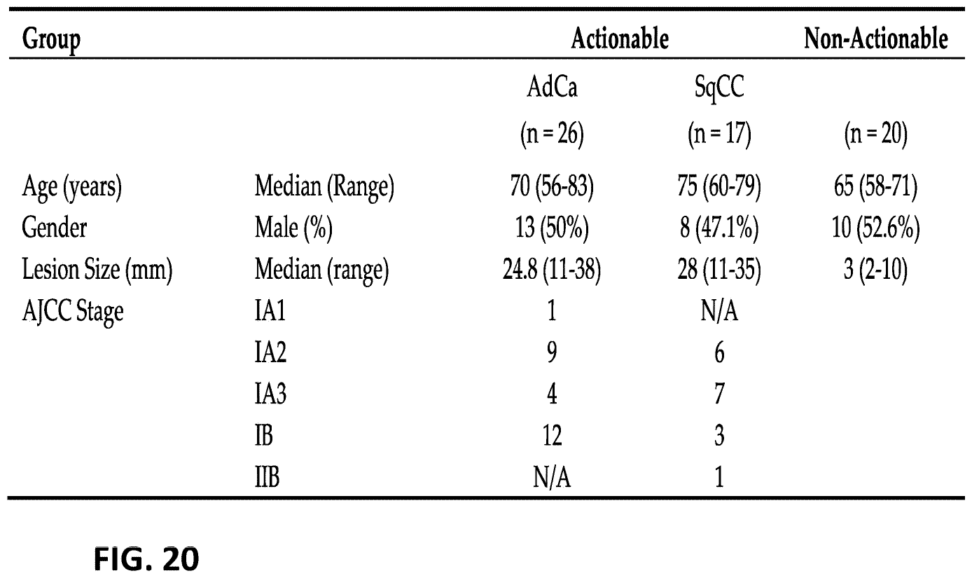

A total of 63 samples were included in the ‘Discovery’ cohort used for the protein microarray study, with patient clinical and demographic information provided (FIG. 20). These samples were combined into 10 sample pools (8 ‘actionable’ and 2 ‘non-actionable), which were utilized to probe the microarrays.

FIG. 20 shows clinical characteristics of the ‘Discovery’ Cohorts used for the HuProt™M protein microarray studies. Basic clinical characteristics of all samples comprising the Discovery Cohort are provided, as used for the HuProt™ microarray studies. The adenocarcinoma (AdCa) and squamous cell carcinoma (SqCC) samples are each divided across 4 sample pools, whereas the “non-actionable” samples are divided across 2 sample pools.

To diversify the coverage of the biomarkers selected, a range of common lung pathologies and malignancies relevant to lung cancer screening were included in this cohort, given the potential these would have unique molecular profiles. Within the lung malignancy cases, all samples included had “actionable” nodules based on Lung-RADS® v1.1 and Lung-RADS® v2022. Additionally, all malignancy cases were confined to T₁₋₃N₀M₀. Briefly, a T₁₋₃N₀M₀ indicates a primary tumor limited to 7 cm and lacking local and distant metastatic progression, respectively. All patients with “non-actionable” nodules qualified for lung cancer screening based on current USPSTF guidelines (i.e., were between the ages of 50-80, had at least a 20 pack-year smoking history, and were current smokers or quit within the last 15 years) at the time of sample collection. A single sample possessing a non-malignant nodule was inadvertently included in one of the ‘non-actionable’groups.

Autoantibodies with Differential Signal in Patients with ‘Actionable’ vs. ‘Non-actionable’ Nodules via HuProt™ Protein Microarrays.

Normalized data from the HuProt™ protein microarrays were processed using empirical Bayesian statistics and displayed with volcano plots to contrast autoantibodies that differentially associate with ‘actionable’ or ‘non-actionable’ nodules (data not shown). Further delineation of the ‘actionable’ group based on histology provided similar plots for AdCa and SqCC ), relative to the ‘non-actionable’ group (data not shown). A total of 501 markers were found to be differentially recognized (p<0.05) in the “actionable” sample pools (AdCa, SqCC, or AdCa and SqCC) compared to “non-actionable” sample pools.

Candidate biomarkers were preferentially selected for further development if the marker was 1) relevant to more than one of the comparison of interest (i.e., AdCa versus high-risk and SqCC versus high-risk), 2) significant after a Benjamini-Hochberg correction (i.e., adjusted p-value), 3) significant in more than one normalization method, and 4) if they were elevated in the “actionable” group compared to the “non-actionable” group.

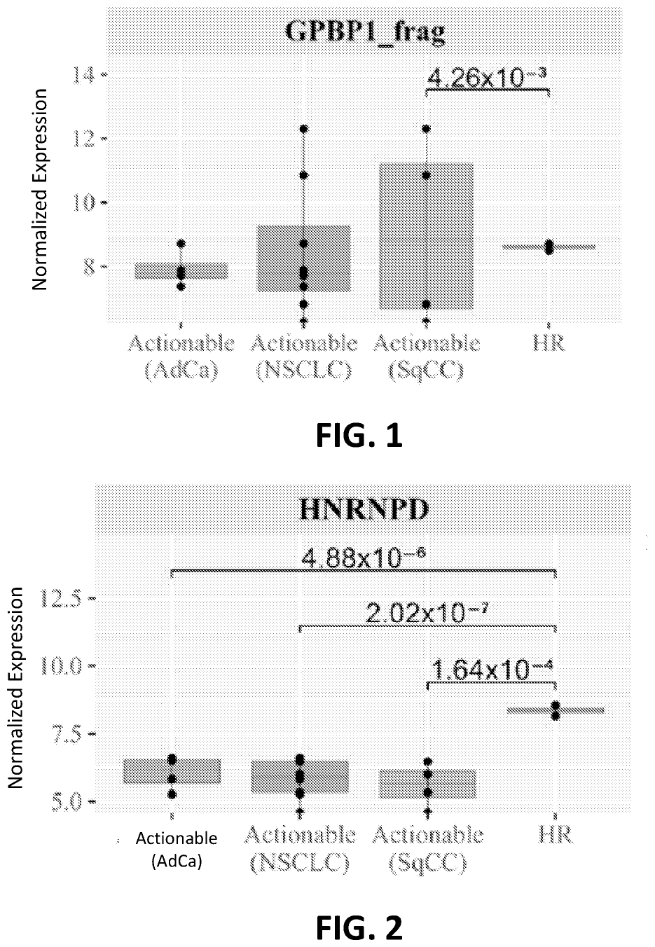

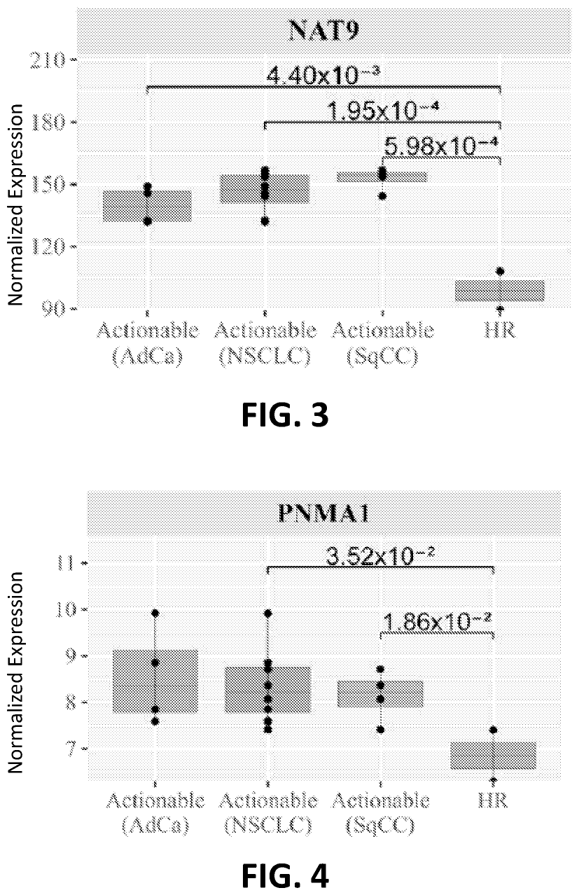

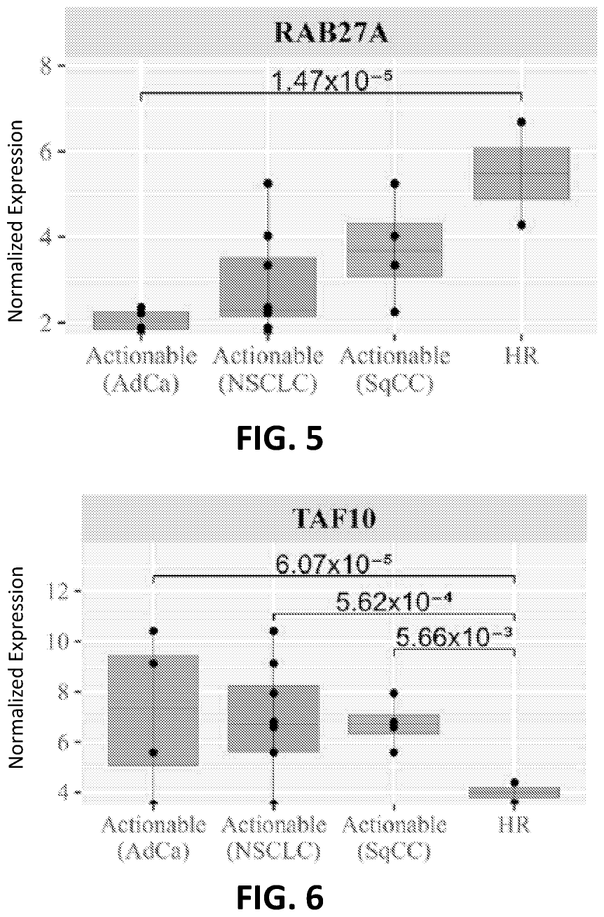

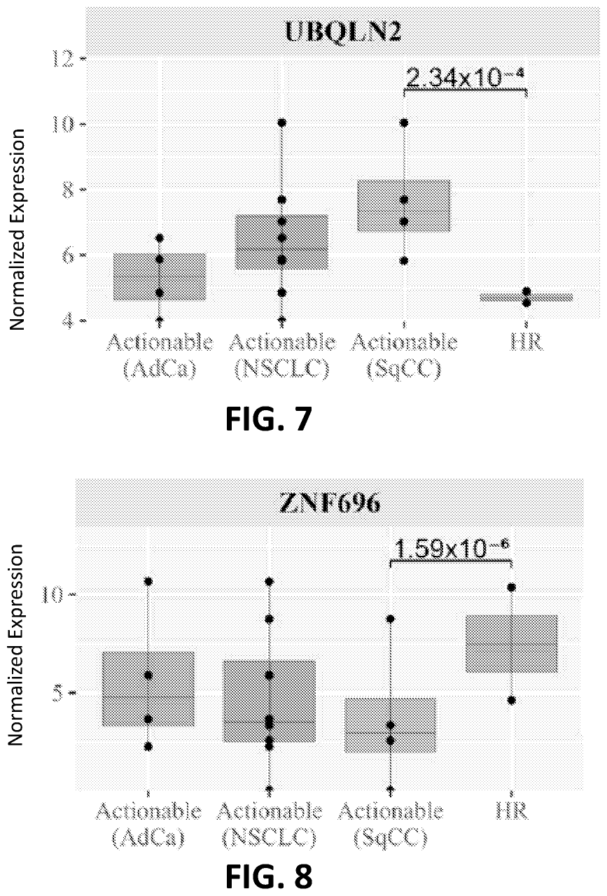

Based on these criteria, eight candidate biomarkers (GPBP1, HNRNPD, NAT9, PNMA1, RAB27A, TAF10, Ubiquillin 2, ZNF696) were selected for development based on ability to distinguish ‘actionable’ from ‘non-actionable’ nodules, with ‘Box and whisker’ plots (or boxplots) for these biomarkers shown in FIGS. 1-8 (FIG. 1-FIG. 8).

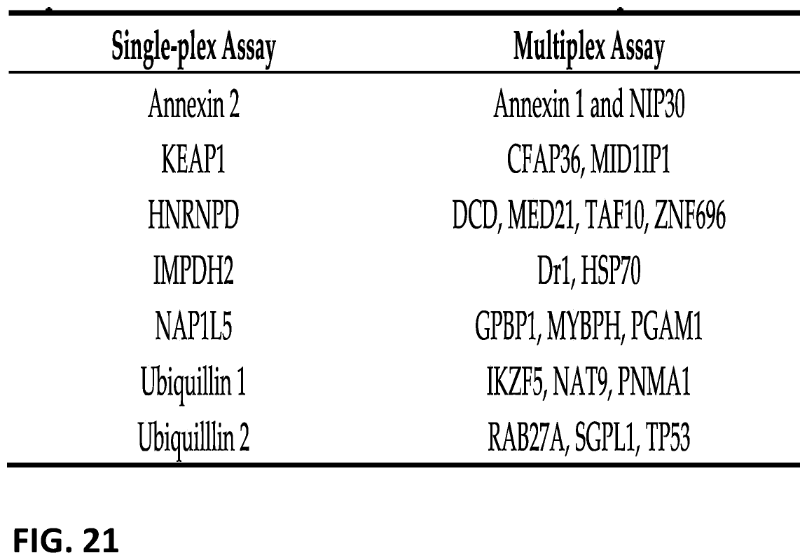

FIGS. 1-8 show Candidate Biomarker Performance for Actionable Versus Non-actionable Nodules within the HuProt™ Microarray. Novel biomarkers for discerning “Actionable” versus “Non-actionable” nodules were determined by comparing autoantibody signal levels between microarrays utilizing empirical Bayesian statistics with the Bioconductor package in R. Adjusted p-values were determined utilizing the Benjamini-Hochberg method. Volcano plots were created for comparisons made between high-risk screening patients with “non-actionable” nodule sample pools and different “actionable” nodule subsets, those with NSCLC (both AdCa and SqCC), AdCa, and SqCC (data not shown). Specific biomarkers selected for further analysis are displayed as boxplots. Inset in each boxplot are numerical values indicating the expression log fold change between actionable nodules (AdCa, NSCLC or SqCC) and non-actionable nodules (HR or High Risk). To maximize the studies coverage of biomarkers, 18 additional biomarkers were assessed which our laboratory found had relevancy to early detection of lung cancer but were not discovered for the express purpose of discerning “actionable” versus “non-actionable” nodules. Utilizing Luminex® assays, we tested a total of 26 different biomarkers within the patient cohort. These biomarkers included the 8 candidate biomarkers discovered via the microarray for discerning “actionable” versus “non-actionable” cohorts, 12 candidate biomarkers with relevance to lung cancer screening questions, and 6 biomarkers which our laboratory had previously tested. Biomarkers were tested with a total of 14 different multiplexes/single-plexes, as shown in FIG. 21.

FIG. 21 shows Candidate biomarkers tested with panel composition indicated. In FIG. 21 each target for which a Luminex® assay was developed and used to test the “Biomarker Development” cohort is listed out. The targets are organized by their multiplex/single-plex group. Multiplexed markers can be measured in tandem within patient serum.

Characteristics of the Biomarker Development Cohort with Subgroups for the Classification Model Development and Assessment Provided.

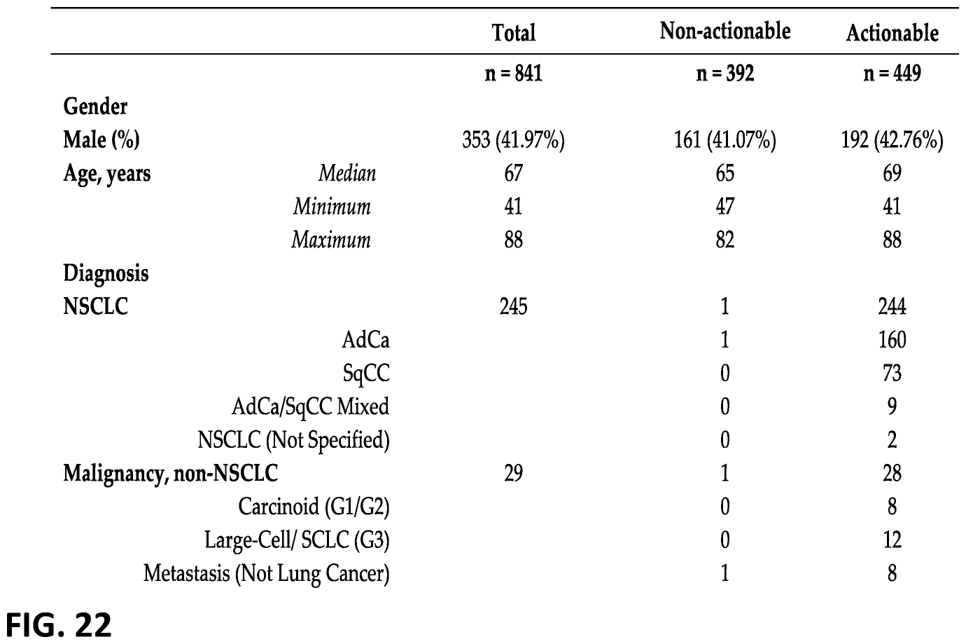

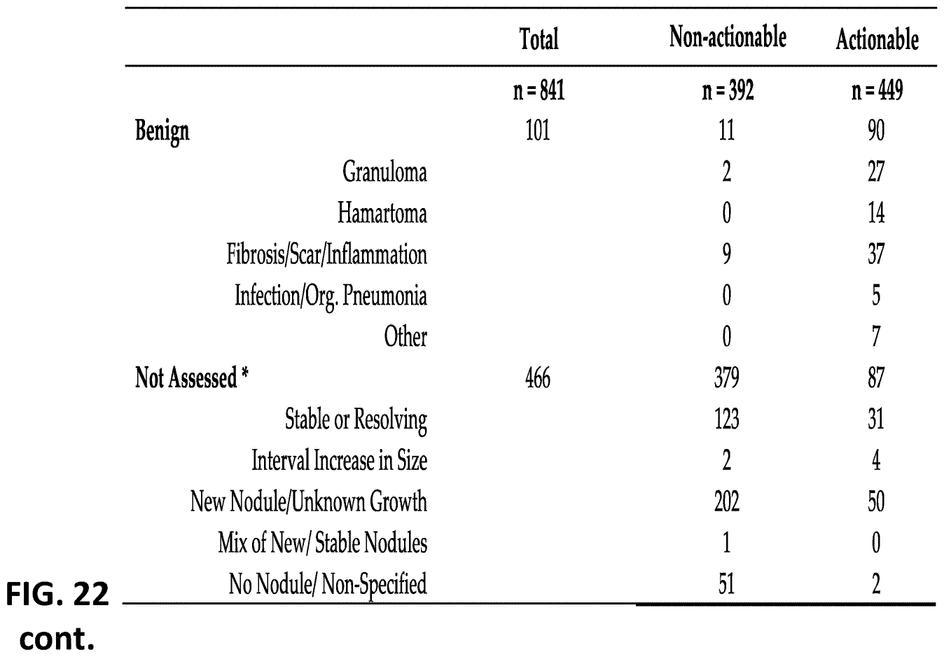

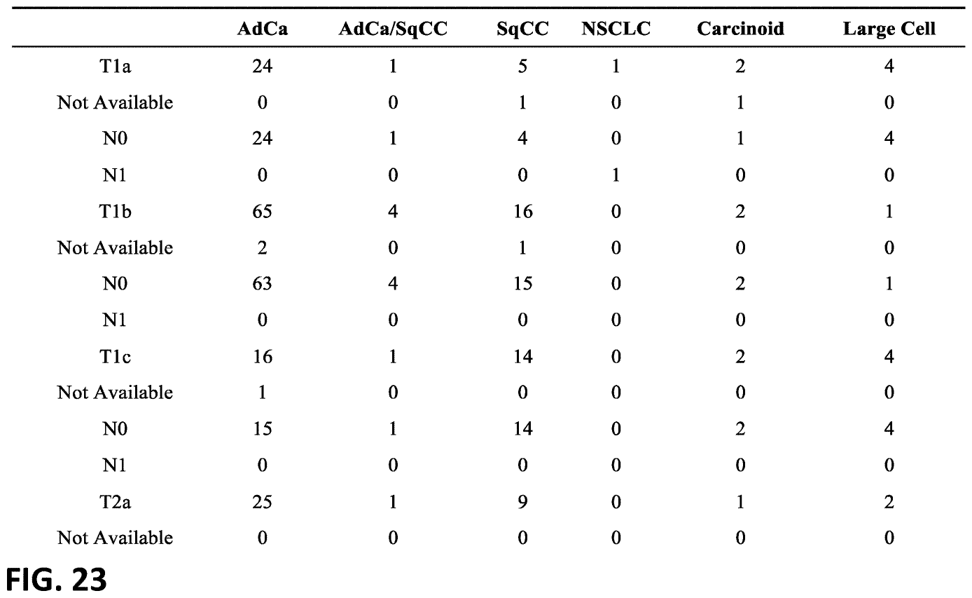

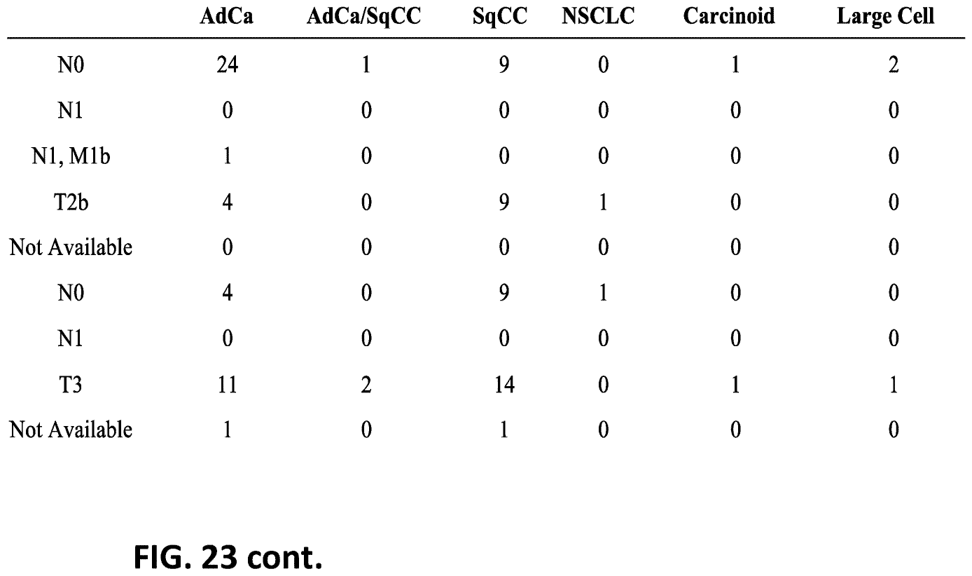

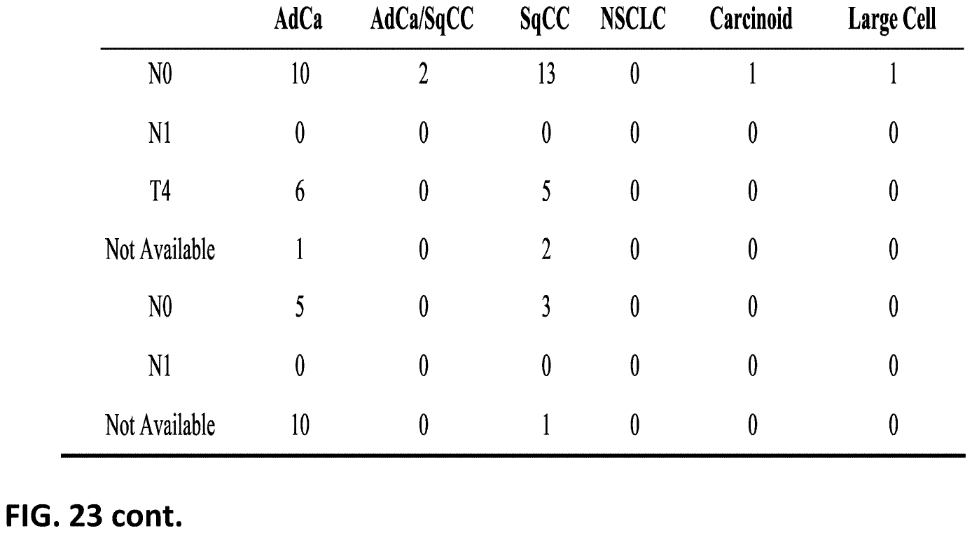

Custom Luminex® assays were used to assess levels of each biomarker within the Biomarker Discovery cohort (n=841). Of the 841 patients within the cohort, 449 patients had “actionable” nodules and 392 patients had “non-actionable” nodules. Histological classifications of the cohort include patients with histologically benign nodules (BN)(n=101), early-stage NSCLC (n=245), high-risk screening (n=466), and patients with other malignancies (n=29) who qualify for screening based on current USPSTF guidelines. Within the lung malignancy samples, a total 265 samples were tested (including Small-Cell Lung Cancer and Carcinoid cases), 162 samples were T_{1a-b}N₀M₀, 52 samples were T_{2a-b}N₀M₀, 29 samples were T₃N₀M₀, and 11 were T₄N₀M₀. Patient breakdowns of this cohort can be seen in FIG. 22. Further breakdown of the nodule development, metastasis, and histological grouping can be seen in FIG. 23.

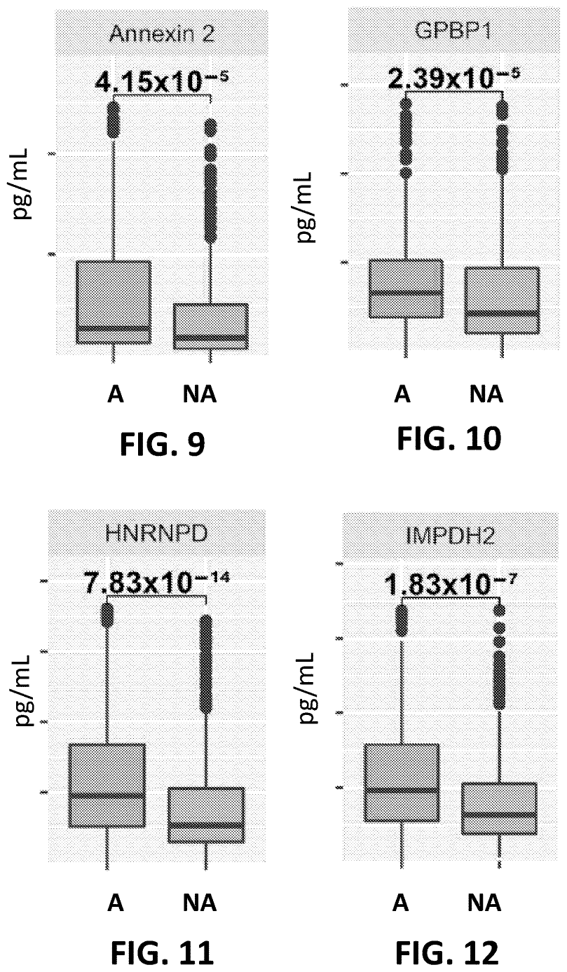

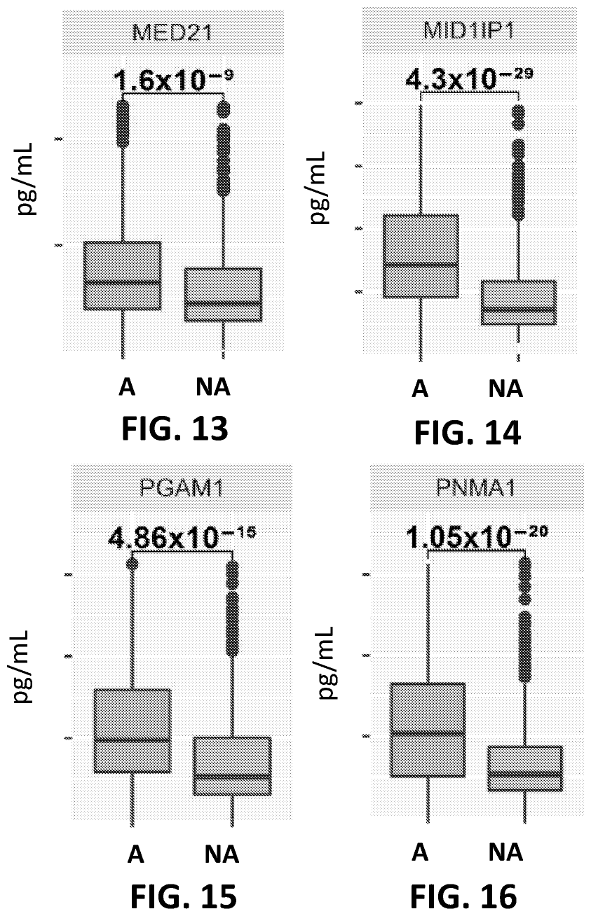

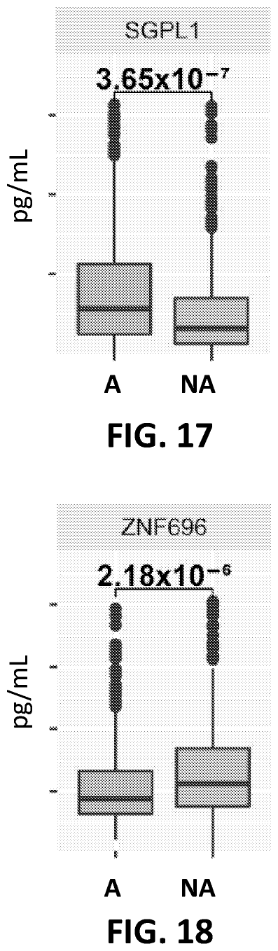

FIG. 22 shows Biomarker Development Cohort. In FIG. 22 the cohort which was tested for the 28 potential biomarkers via Luminex® assays is broken down by patient demographics. Of the 26 candidate biomarkers tested, 17 were significant for discerning “actionable” cases from “non-actionable” cases, with p-values≤0.05. 12 biomarkers were significant with p-values <0.01. Looking specifically at the performance of the 8 novel markers discovered utilizing the HuProt™ microarray (GPBP1, HNRNPD, NAT9, PNMA1, RAB27A, TAF10, Ubiquillin 2, ZNF696) for discerning “actionable” versus “non-actionable” nodules, 7 were significant with p-values <0.05, and 5 had p-values <0.01. Performance for all 26 biomarkers tested are listed in FIG. 24, whereas boxplots for the top 10 most significant markers shown in FIG. 9-FIG. 18.

FIG. 24 shows Performance of 26 Biomarkers for Discerning Between the “Actionable and “Non-actionable” Cohorts. Individual biomarker performances were compared between “actionable” and “non-actionable” nodules via Mann-Whitney U Test.

FIGS. 9-18 : Boxplots for the Highest Performing Biomarkers for Discerning Between the “Actionable and “Non-actionable” Cohorts. The top 10 most significant biomarkers of the 26 biomarkers assessed within the large cohort are shown as ‘box-and-whisker’ plots (or boxplots).

### Performance of Logistic Regression Produced From Top Biomarkers

After determining the significance of each marker, we wanted to ascertain each markers individual ability to discern between “actionable” and “non-actionable” cases based on a generalized linear models. Logistic regression models were trained for each of the top 10 most significant biomarkers based on 60% of the total collected data, with 40% of the data left for a testing set (data not shown). Performance metrics were determined for the generalized linear models, for both the training and testing set based on the optimal cut-off determined from the training set. AUCs for the top biomarkers ranged from 0.58-0.72, with MID1IP1 having the highest AUC (0.72). Interestingly, while MID1IP1 had the highest AUC, MED21 had the highest sensitivity with 79% sensitivity in the training cohort and 83% sensitivity in the testing. While accuracy is an important metric for determining models performance, due to the nature of the test we are developing, high sensitivities are of higher importance as false-negatives result in delayed diagnosis of cancer, whereas a false-positive leads to test follow-up through LDCT.

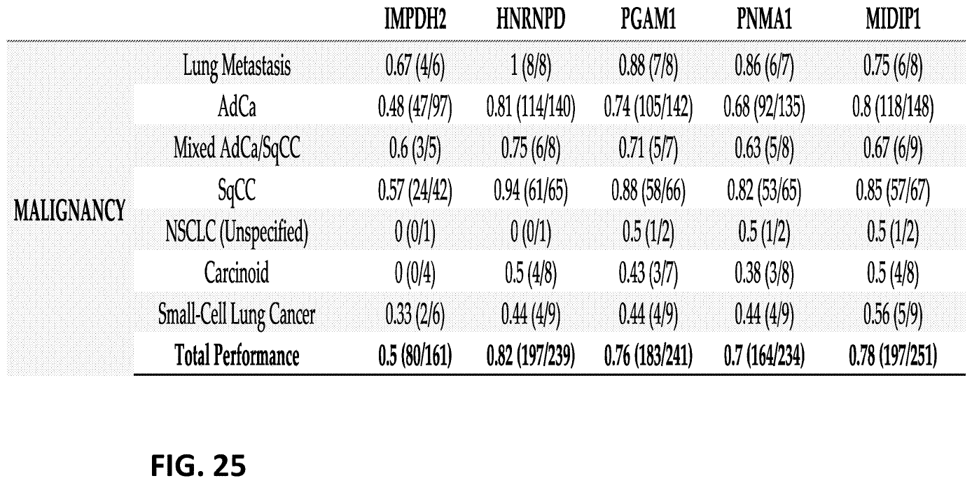

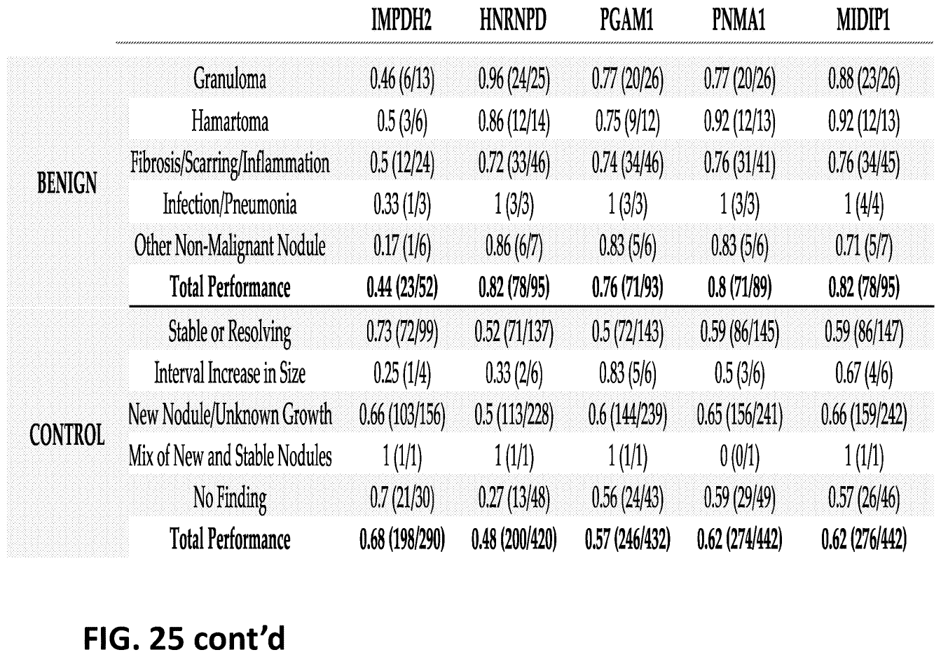

To further assess the performance of the individual biomarkers we broke down the performance by clinically distinct cohorts: benign cohort, malignant, or high-risk screening cohort. Breakdowns by total accuracy in classification can be seen in FIG. 25 for the top 5 most significant biomarkers. For lung malignancy the accuracy of the models based on the individual biomarkers ranged from 50%-82%, with the best marker being HNRNPD. For histologically benign cases the accuracy was 44%-82%, with HNRNPD and MID1IP1 both having an “actionable” classification accuracy of 82%. Finally, for the screening cohort the classification of samples into “actionable” or “non-actionable” subsets was 45-68% with the highest accuracy within the IMPDH2 biomarkers.

Creation of Random Forest Model for Determining Actionable versus Non-actionable Nodules

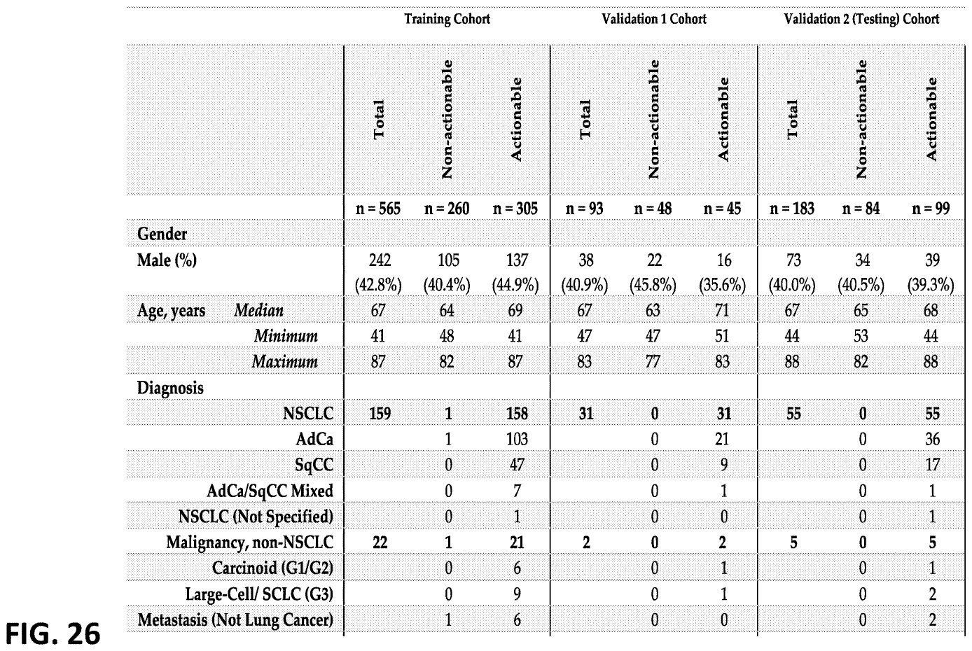

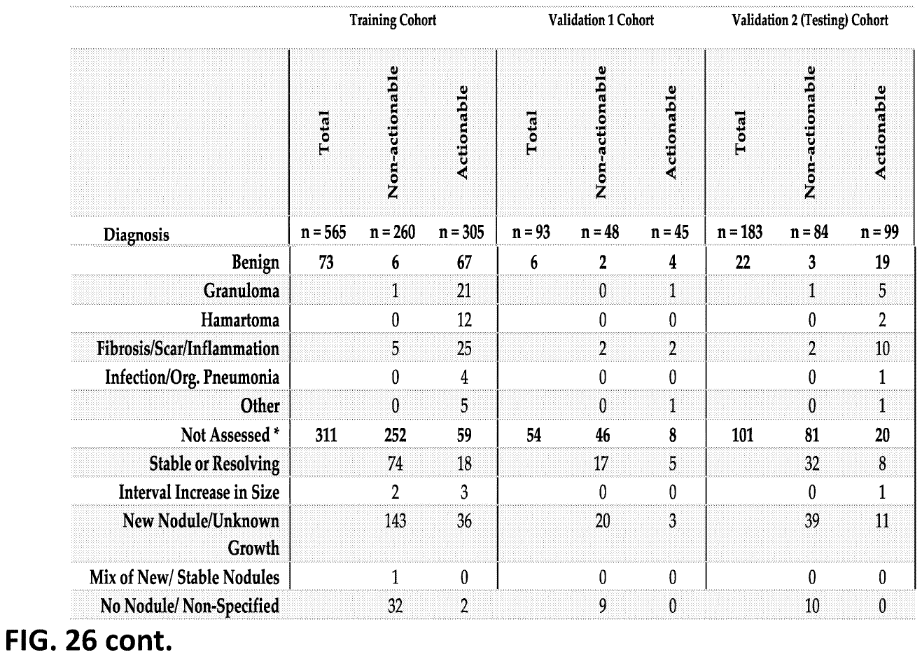

After determining the performance of the individual biomarkers, we aimed to develop a panel of biomarkers to obtain optimized performance characteristics for identifying patients with “actionable” versus “non-actionable” nodules. For this, our Biomarker Development Cohort was subdivided into Training, Validation 1, and Validation 2 sub-cohorts, as described earlier and with characteristics shown in FIG. 26.

Machine learning models have been shown to help improve overall performance of biomarker panels and have become standard for the purpose of developing clinical biomarker tests. Our laboratory established a panel in which we utilized a Random Forest machine learning model to develop a model with good sensitivity and specificity for discerning between four clinically distinct groups: early-stage lung cancer, osteoarthritic, non-neoplastic nodules, and COPD/asthma patients. Basing off of this, we decided to attempt to develop a random forest model for discerning between “actionable” and “non-actionable” nodules.

### Development of a Preliminary Biomarker Panel via Machine Learning

The objective of this step was to determine the optimal combination of biomarkers for risk-stratifying patients for potential identification of an actionable nodule via LDCT-based screening protocols. For feature selection, we calculated performance for every unique combination of 6 and 7 biomarkers of the 19 biomarkers identified from our microarray and from our previously published panel. Seven biomarkers were eliminated from analysis (DR1, IMPDH2, NAT9, IKZF5, KEAP1, RAB27A, Ubiquillin 2) due to >20% missing data-based on percent coefficient of variation and low bead counts. For the rest of the biomarkers, imputation was used within the training cohort to maximize the number of samples that could be used for model development. We examined each combination of six and seven biomarkers, training a random forest model and recording its performance metrics seen within the Training and Validation 1 cohort. The exploration with all possible six marker combinations tested a total of 27,132 different combinations, and the one with all possible seven marker combinations tested a total of 50,388 different combinations. The top outputs of each six and seven marker combinations were compared, and given the accuracy was less than 2% different between the models, we opted to utilize the 6 marker model to economize future studies with this panel.

The panel which showed the greatest accuracy within the Validation 1 cohort was Annexin 2, DCD, MID1IP1, PNMA1, TAF10, and ZNF696. Three of these biomarkers (PNMA1, TAF10, and ZNF696) were chosen based on the HuProt™ microarray discovery outlined in sections 3.1 and 3.2. Annexin 2 was part of a biomarker panel our laboratory had previously published, with DCD and MID1IP1 both holding value for early detection based in previous laboratory studies. Of the biomarkers chosen, four (MIDIP1, PNMA1, Annexin 2, and ZNF696) had individual p-values <0.01, one (TAF10) had a p-value <0.05, and, interestingly, one (DCD) was not significant (p>0.05), for discerning “actionable” from “non-actionable” cases in the Biomarker Development cohort. We further tested the panel on the Validation 2 cohort which was not utilized for panel determination or optimization purposes. The Validation 2 cohort consisted of 183 patients, with 84 patients at high-risk of lung cancer with “non-actionable” nodules and 99 patients with “actionable” nodules (solid pulmonary nodules >6 mm). The accuracy within the third cohort was 72.48%, with a sensitivity of 76.62% and specificity of 68.06%.

Performance of Final Optimized Panel for Patient Risk Stratification Given the objective of this study was to develop a risk stratification tool to pre-screen individuals for LDCT-based lung cancer screening protocols, we aimed to focus on minimizing ‘false-negative’ findings to reduce the potential of missing a malignancy at the cost of some ‘false-positives’. To this end, we created an ROC curve on the Validation 1 cohort. Assessing different cut-points used for the ROC curve, we selected the cut-off which had >95% sensitivity within the Validation 1 cohort with the least cost to specificity. The resulting risk cut-off was 0.334 as opposed to the standard 0.5 which the model was originally trained.

The performance characteristics were recalculated within the Validation 1 and Validation 2 cohort (data not shown). Within the Validation 1 and Validation 2 cohorts, sensitivity was high, at 97.2% and 93.5% respectively. With the increase in sensitivity there was a decrease in specificity to 50% and 36.1% respectively. The accuracy of the model was tested against different racial and gender breakdowns (data not shown). Notably, negative predictive values (NPVs) between white and African American subgroups were comparable.

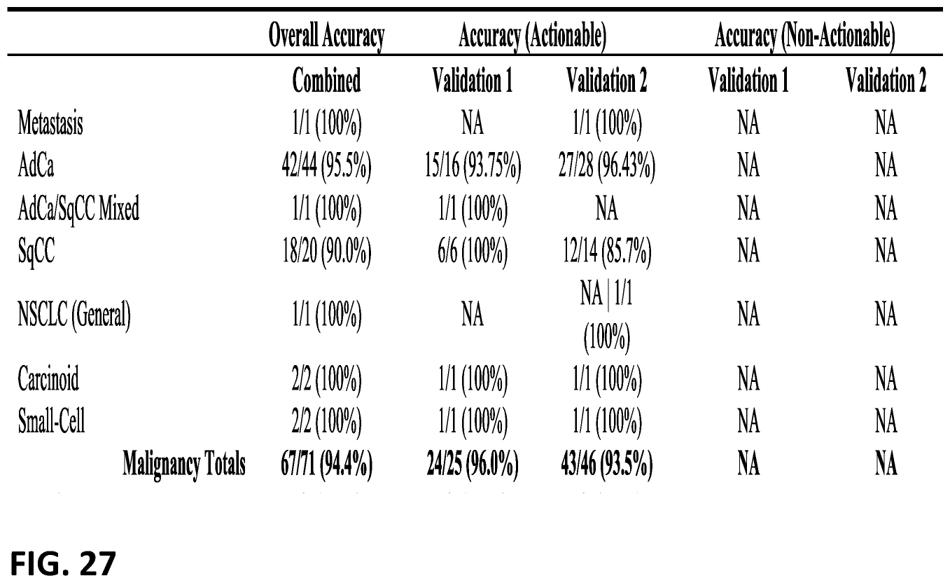

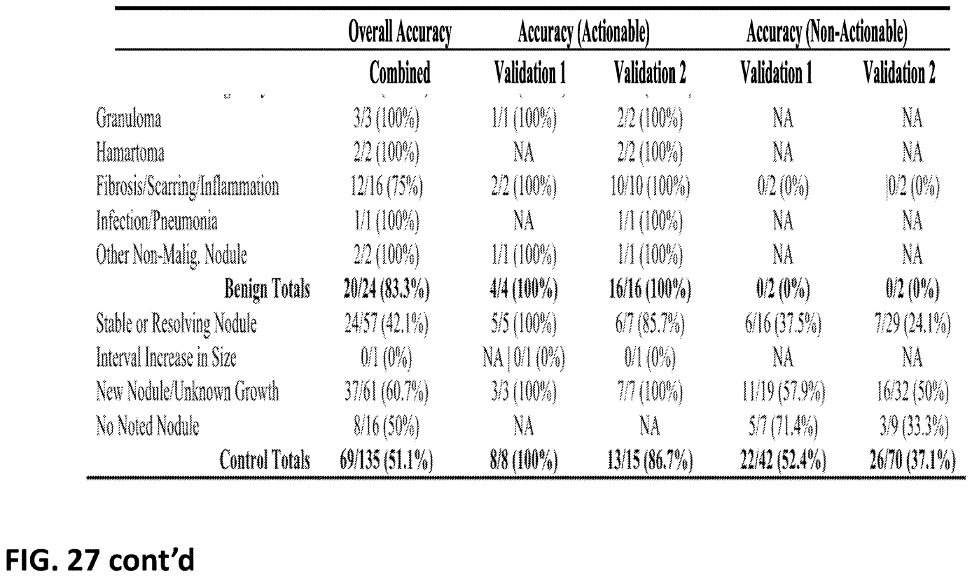

The panel was further assessed for its performance for different clinically distinct subgroups. The Validation 1 and Validation 2 cohorts were further subdivided into three main groups: malignancy cases, histologically benign cases, and high-risk screening cases. These subgroups were then evaluated based on disease and nodule presentation. The accuracy of the classification of each of these subgroups are shown in FIG. 27.

FIG. 27 shows Performance of Sensitivity Optimized Model broken Down by Clinically Distinct Cohort. Performance characteristics of the sensitivity optimized biomarker panel is broken down by clinically distinct groups.

For the malignancy cases (n=71), there was an overall classification accuracy of 94.4%, with all malignancy cases designated as “actionable” nodules (i.e., >6 mm). Performance was high across all subsets of lung malignancy with ranges from 90-100% accuracy. In the future, it may be of interest to assess additional biomarkers which hold value for discerning SqCC, to help supplement the current panel and improve performance within this subset of patients.

Histologically benign cases are made up of serum samples obtained from patients who underwent lung resection and were determined to have “benign” nodules by pathology. Within this cohort there was high accuracy of 83.3%. Notably, within the benign cases the “non-actionable” cases were the only cases which were misclassified. This suggests that this panel may perform very well at identifying patients with a benign lung disease.

Finally, we assessed the performance in the high-risk screening cohort. The high-risk screening cases made up the majority of the “non-actionable” cases assessed in this study. Of the screening cohort, 51.1% of the cohort was properly classified as having “actionable” or “non-actionable” nodules. The performance within the screening cohort with “actionable” nodules was high, with 21/23 samples within the Validation 1 and Validation 2 properly classified. Within the screening cohort with “non-actionable” nodules performance was worse, with only 48/112 cases being identified as “non-actionable”. This is likely a result of the panel being optimized for the identification of “actionable” nodules, with false-positives having less of an importance due to the availability of other testing mechanisms. Additionally, for 56 of the 64 misclassified “non-actionable” cases, subsequent LDCT screening results were available and assessed. During subsequent screens, 6/56 had “actionable” nodules, with 3 being classified as 4A, 2 classified as 4B, and 1 classified as 3 via Lung-RADS® v 1.1 or v 1.0 depending on date of screening. Taking these cases into account, this would potentially increase the specificity of this test to 54.5% in the Validation 1 cohort and 41.7% in the Validation 2 cohort.

### Discussion

NSCLC is the leading cause of cancer related mortality world-wide, much of which can be attributed to late diagnosis. Screening is key to detecting malignancies early, and the current mechanism for screening is annual LDCTs within high-risk populations. While LDCT can be extremely sensitive for the detection of lung nodules, with estimates reported from 59% to 100%, specificity of LDCT tends to be lower with estimates which can vary dramatically from 26.4% up to 99.7%. These detection ranges highlight the variability with this screening method, as training of radiologists may dramatically impact test performance. Improved methods for identifying patients of interest for LDCT screening, based on circulating biomarkers, might help enhance the specificity of the overall lung cancer screening paradigm. To this end, we developed a blood-based biomarker test which could aid in the identification of patients with “actionable” nodules. It is estimated that only about 10-20% of high-risk patients will have actionable nodules, thus many unnecessary (or ‘futile’) LDCTs may be avoided, and ‘false-positive’ rates may decrease if we are able to identify these patients. A simple blood-based test would be much more accessible, compared to LDCT, thus increasing the convenience for the initial evaluation (a ‘pre-screen) and potentially increasing uptake rates, which are currently low (6-14%) among patients. We have developed a blood-based test which has a sensitivity of 93.5%, with a specificity of 36.1%. According to multiple studies, approximately 80-90% of patients who qualify for screening do not harbor “actionable” nodules. This means that our test could decrease the number of futile scans by approximately 30%, while maintaining the sensitivity levels observed in LDCT.

Our efforts to identify markers that held value for discerning patients with “actionable” versus “non-actionable” nodules were focused on the discovery of circulating autoantibodies. To avoid autotoxicity, B-cells generally have a central and peripheral tolerance, which prevents them from producing autoantibodies targeted at self-antigens. However, it is believed that B-cells can overcome their peripheral tolerance when tumors produce autoantigens that are overly expressed, expressed in areas of the body they typically are not, or are a result of a mutation that leads to an antigenic (or neoantigenic) protein structure. Thus, studies have noted the presence of autoantibodies within cancer and at very early stages of tumor development, making them ideal screening biomarker candidates.

Multiple papers have focused on the development of a blood-based autoantibody biomarker tests for lung malignancy detection. Our laboratory established an autoantibody panel which consisted of Annexin 1, Annexin 2, IMPDH2, HSP70, PGAM1, and Ubiquillin 1, and properly classified 93% within 5 clinically distinct groups: osteoarthritis, “cancer-free” control, asthma/COPD, benign nodule, and NSCLC. These markers were included within this study.

### Conclusions

With the completion of this study, we have identified biomarkers for the purposes of discerning “actionable” versus “non-actionable” nodules, utilizing protein microarrays. We then determined individual biomarker performance metrics for discerning between “actionable” and “non-actionable” cohorts based on general linear models. Finally, we developed a machine learning model based on circulating levels of 6 biomarkers (Annexin 2, MID1IP1, PNMA1. TAF10, and DCD), which holds high sensitivity (97.2% in validation 1 and 93.5% in validation 2) for the purposes of identifying patients with “actionable” nodules.

Within the eligible screening population, only about 10-20% have “actionable” pulmonary nodules. Patients with “actionable” nodules have an increased risk of 9-96 times greater for developing lung cancer compared to the rest of the high-risk screening cohorts. Those at high-risk without “actionable” nodules are estimated to have a similar lung cancer rate, 0.1%, as never smokers with estimates of 0.1% in females and 0.2% in males. This suggests that our biomarker test could identify a small population (10-20%) within the general screening population, who would benefit from LDCT. The majority of the screening patients may only need the annual simple, cost-efficient, and more easily accessible biomarker blood test. This panel of biomarkers may help improve the current initial lung cancer screening paradigm. In future, more validation studies within a larger patient cohort consisting of high-risk screening patient will be of interest for model optimization on large populations with more reflective population breakdowns.

While embodiments have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

As will be appreciated from the descriptions herein, a wide variety of aspects and embodiments are contemplated by the present disclosure, examples of which include, without limitation, the aspects and embodiments disclosed herein.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of detecting an actionable lung nodule in a subject comprising:
contacting a biological sample obtained from the subject comprising an autoantibody biomarker with a target protein,
detecting the binding of the target protein with the autoantibody biomarker which is specific to the target protein;
measuring a level of the autoantibody biomarker present in the biological sample; wherein the level is indicative of an actionable lung nodule when compared to a reference sample.

2. (canceled)

3. The method of claim 1 , wherein the subject is a human.

4. (canceled)

5. (canceled)

6. (canceled)

7. (canceled)

8. (canceled)

9. (canceled)

10. (canceled)

11. (canceled)

12. (canceled)

13. (canceled)

14. The method of claim 1, wherein the biological sample is selected from the group consisting of whole blood, plasma, serum, bronchial lavage fluid, sputum, saliva, urine, amniotic fluid, lymph fluid, tissue or fine needle biopsy samples, peritoneal fluid, cerebrospinal fluid, and includes supernatant from cell lysates, lysed cells, cellular extracts, and nuclear extracts.

15. (canceled)

16. (canceled)

17. (canceled)

18. (canceled)

19. The method of claim 1, wherein the target protein is selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, MID1P1, DCD, MED21, TAF10, ZNF696, DR1, HSP70, GPBP1, MYBPH, PGAM1, IKZF5, NAT9, PNMA1, RAB27A, SGPL1, and TP53.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (Canceled)

47. (canceled)

48. The method of claim 1 further comprising measuring a panel of N target proteins, wherein N=1 to 26, and wherein each target protein is specific to an autoantibody.

47. The method of claim 48, wherein the target proteins are selected from the group consisting of ANNEXIN 2, KEAP1, HNRNPD, IMPDH2, NAP1L5, UBIQUILLIN 1, UBIQUILLIN 2, ANNEXIN 1, NIP30, CFAP36, MID1P1, DCD, TAF10, MED21, ZNF696, DR1, HSP70, GPBP1, MYBPH, PGAM1, IKZF5, NAT9, RAB27A, SGPL1, and TP53. (Currently Amended) The method of claim 1, wherein detection of the autoantibody biomarker is performed using a detection method selected from mass spectrometry, immunoassay, electrochemical luminescent assay, electrochemical voltametry, electrochemical amperometry, atomic force microscopy, radio frequency multipolar resonance spectroscopy, confocal microscopy, non-confocal microscopy, fluorescence optical detection, luminescence optical detection, chemiluminescence optical detection, absorbance optical detection, reflectance optical detection, transmittance optical detection, birefringence optical detection, refractive index detection, surface plasmon resonance, ellipsometry, resonant mirror detection, grating coupler waveguide detection, interferometry, or a combination thereof.

51. The method of claim 1, wherein the reference sample is provided from a pool of healthy individuals or from a pool of non-actionable lung nodule samples.

52. The method of claim 1, wherein the actionable lung nodule is indicative of lung cancer.

53. The method of claim 52, wherein the lung cancer is selected from the group consisting of carcinoid, adenocarcinoma (AdCa) and squamous cell carcinoma (SqCC).

54. (canceled)

55. (canceled)

56. The method of claim 1 further comprising administering a therapeutic effective amount of a cancer treatment.

57. The method of claim 56, wherein the cancer treatment is configured to treat a lung cancer.

58. The method of claim 56, wherein the cancer treatment is selected from surgery, chemotherapy, radiotherapy, immunotherapy, cancer vaccine, or a combination thereof.

59. (canceled)

60. (canceled)

61. (canceled)

62. An autoantibody biomarker panel comprising N target proteins, wherein N=1 to 26, and wherein each target protein is specific to an autoantibody for the detection of an actionable lung nodule.

63. The panel of claim 62, wherein the target proteins are selected from ANNEXIN 2, KEAP1, HNRNPD, IMPDH2, NAP1L5, UBIQUILLIN 1, UBIQUILLIN 2, ANNEXIN 1, NIP30, CFAP36, MID1P1, DCD, TAF10, MED21, ZNF696, DR1, HSP70, GPBP1, MYBPH, PGAM1, IKZF5, NAT9, RAB27A, SGPL1, and TP53.

64. A kit comprising a biomarker panel according to claim 63.

65. (canceled)

66. (canceled)

67. (canceled)

68. (canceled)

69. A method of diagnosing an actionable lung nodule in a subject comprising:
contacting a biological sample obtained from the subject comprising an autoantibody biomarker with a target protein, which is specific to the autoantibody, to form a bound autoantibody;
contacting the bound antibody with a detection molecule;
determining a level of the bound autoantibody present in the biological sample;
providing a diagnosis based on the level of the bound autoantibody biomarker compared to a reference sample, wherein an altered level is indicative of an actionable lung nodule.

70. (canceled)

71. (canceled)

72. (canceled)

73. The method of claim 69 further comprising administering a therapeutic effective amount of a cancer treatment.

74. The method of claim 73, wherein the cancer treatment is configured to treat a lung cancer.

75. The method of claim 74, wherein the cancer treatment is selected from surgery, chemotherapy, radiotherapy, immunotherapy, cancer vaccine, or a combination thereof.
